(19) United States
(12) Patent Application Publication
(10) Pub. No.: US 2020/0330626 A1
(43) Pub. Date: Oct. 22, 2020

(54) FLUORINE-18 LABELED COMPOSITIONS AND THEIR USE IN IMAGING OF BIOLOGICAL TISSUE

(71) Applicant: CORNELL UNIVERSITY, Ithaca, NY (US)

(72) Inventors: Richard Ting, New York City, NY (US); Omer Aras, New York, NY (US); Ye Wang, New York City, NY (US)

(21) Appl. No.: 16/654,783

(22) Filed: Oct. 16, 2019

Related U.S. Application Data
(63) Continuation-in-part of application No. PCT/US2019/027864, filed on Apr. 17, 2019.

(51) Int. Cl. A61K 51/12; A61K 49/00

(52) U.S. Cl. CPC A61K 51/1203; A61K 49/0032; A61K 49/0043

(57) ABSTRACT

A method for internal imaging of biological tissue in a subject by positron emission tomography (PET) or single photon emission computer tomography (SPECT), the method comprising: (i) administering to a subject an imaging agent that includes, at minimum, at least one fluorine-18 radionuclide bound directly or indirectly to a fluorophore, and (ii) imaging internal biological tissue of the subject by PET or SPECT. In further embodiments, the method includes (i) administering to a subject an imaging agent that includes at least one fluorine-18 radionuclide bound directly or indirectly to a fluorophore, and at least one biological entity (e.g., blood cell, peptide, nucleotide, aptamer, targeting agent, antibody, or antibody fragment) bound directly or indirectly to the fluorophore; and (ii) imaging internal biological tissue of the subject by PET or SPECT. In some embodiments, the method further includes simultaneously imaging the internal biological tissue by fluorescence imaging.

### CROSS REFERENCE TO RELATED APPLICATIONS

This application claims the benefit of priority from International Patent Application No. PCT/US2019/027864, filed on Apr. 17, 2019, and U.S. Provisional Application No. 62/658,880, filed on Apr. 17, 2018, which are herein incorporated by reference in their entirety.

### GOVERNMENT SUPPORT

This invention was made with government support under grant number EB013904 awarded by the National Institutes of Health. The government has certain rights in the invention.

### FIELD OF THE INVENTION

The present invention generally relates fluorine-18 labeled compositions, and methods of synthesis and use as cell labeling reagents, and more particularly, to fluorophore compositions containing fluorine-18 and methods of using such compositions for cell labeling and biological imaging by positron emission tomography (PET).

### BACKGROUND OF THE INVENTION

The relationship between imaging and the emergency management of stroke is extremely important given that ischemic stroke can be reversed with tissue plasminogen activator (tPA). Unfortunately, the incorrect administration of tPA to a patient with a hemorrhagic stroke can diametrically worsen a patient's prognosis, making an inaccurate diagnosis of ischemic stroke particularly lethal. The benefits of tPA in treating ischemia diminish if administration is a few hours late, presenting an additional, time-dependent complication to the dilemma of tPA administration. The need to quickly and accurately diagnose intracerebral hemorrhage (ICH) in patients that present with symptoms of stroke highlight the importance of imaging in brain injury.

Hemorrhage in the brain is best imaged with contrast. Cerebral computed tomography angiography (CTA, Iodine contrast computed tomography (CT)) and magnetic resonance angiography (MRA, gadolinium contrast magnetic resonance imaging (MR)) can be used to clearly visualize the vessels and blood pool associated with hemorrhage. Unfortunately, added risk accompany CT and MR contrast imaging including thrombus formation, renal failure, shellfish allergies (CTA), and the presence of paramagnetic implants (MRA). Patients must be first evaluated for contraindications, which take time, and inevitably, not all patients can receive CTA or MRA. For these reasons, contrast imaging is generally not performed in emergencies. Lower resolution, non-contrast CT is currently the standard, and a key indication for determining hemorrhage and intravenous tPA administration in acute brain injury. Non-contrast CTs do not always provide adequate resolution for a clinician to clearly diagnose an infarction. In these cases, the decision to administer tPA to a patient suffering from ischemic stroke can be delayed for a second opinion or additional scanning While a delay can be unfortunate to a patient suffering from ischemic stroke, inaction is preferred to the added trauma and liability that accompany a clinician's decision to administer tPA to a patient bearing a poorly-imaged, misdiagnosed, hemorrhagic stroke.

The ability to improve prognosis should not be sacrificed because of inadequate resolution. Positron emission tomography (PET) is a technique that does not have life-threatening contraindications. PET is also directly compatible with CT, and scanners that can simultaneously acquire PET and CT data are becoming common place in clinical settings because of the popularity of [¹⁸F]-fluorodeoxyglucose (FDG)-PET. Unlike CT and MR contrast, [¹⁸F]-PET tracers for imaging hemorrhage and blood pool can be useful in emergency, acute stroke situations, and patients with compromised kidney function can be imaged by [¹⁸F]-PET. PET and non-contrast CT can be acquired simultaneously, without the need to consider life-threatening contraindications.

The best agent for imaging hemorrhage is, unironically, blood. Unlike small molecules, labeled blood cells can never incorrectly indicate hemorrhage by occupying a blood-free-space. Changes in blood flow occur before metabolic and oxic changes in tissue, making the red blood cell (RBC) superior to [¹⁸F]-FDG and [¹⁸F]-hypoxia sensors for imaging ICH acutely, in timeframes that could affect tPA administration. [^{99m}Tc]-labeled red blood cells (RBCs) and [^{99m}Tc]- leukocytes (exametazime) are currently used clinically to image stroke and intestinal/renal bleeding using single photon emission computed tomography (SPECT). However, due to their low resolution, conventional labeled blood cells do not as yet have the ability to image intracranial hemorrhage.

### SUMMARY OF THE INVENTION

The present invention provides novel ¹⁸F-labeled blood cells for imaging of biological tissue by positron emission tomography (PET) or single photon emission computer tomography (SPECT), although, notably, [¹⁸F]-PET is generally superior to SPECT because it can be used at lower doses and acquired at superior resolutions. The ¹⁸F-labeled blood cells described herein also possess the advantage of being imaged by fluorescence imaging. More specifically, the method described herein includes the following steps: (i) administering to a subject an imaging agent that includes, at minimum, at least one fluorine-18 radionuclide bound directly or indirectly to a fluorophore, and (ii) imaging internal biological tissue of the subject by PET or SPECT. In further embodiments, the method includes (i) administering to a subject an imaging agent that includes at least one fluorine-18 radionuclide bound directly or indirectly to a fluorophore, and at least one biological entity (e.g., blood cell, peptide, nucleotide, aptamer, targeting agent, antibody, or antibody fragment) bound directly or indirectly to the fluorophore; and (ii) imaging internal biological tissue of the subject by PET or SPECT. In some embodiments, the method further includes simultaneously imaging the internal biological tissue by fluorescence imaging.

The imaging method described herein advantageously provides the superior resolutions and quick diagnoses afforded by PET or SPECT while at the same time avoiding the risks associated with use of contrast agents commonly used in other imaging methods, such as computed tomography (CT), magnetic resonance imaging (MRI), and magnetic resonance angiography (MRA). The imaging method described herein achieves this by (i) administering to a subject blood cells that have been labeled with a positron-emitting fluorescent imaging compound containing at least one fluorine-18 radionuclide bound directly or indirectly to a fluorophore; and (ii) imaging internal biological tissue of the subject by PET or SPECT. By virtue of the presence of a fluorophore in the label, the method can advantageously image internal biological tissue by clinical fluorescence imaging, in addition to PET, if desired. The method finds use in PET imaging for assessing or monitoring a range of conditions, such as, for example, transplant rejection or acceptance, the extent or progression of a cancer, or the extent or progression of a hemorrhage.

Some unique features of the invention include: 1) fluoridation on and at a non-carbon bearing molecule that can be used to stably radiolabel a cell and show imaging of cells by PET in vivo, 2) the ability to image radiolabeled cells by fluorescence, which can be used to confirm that the radiolabel does not transfer between cells and to image bleeding by fluorescence, and 3) use in an emergency bleeding situation. The [¹⁸F]-RBCs describe herein are superior to its counterpart RBC imaging agents including; pre-clinical chromium and gadolinium RBCs (MR contrast), and current, clinical SPECT agents [^{99m}Tc]-RBC and [^{99m}Tc]- leukocyte (exametazine) because of the higher resolution, lower quantity, and lower activities at which [¹⁸F]-RBCs can be imaged. As further discussed later in this disclosure, the superior imaging potential of [¹⁸F]-RBCs can be used to image lesions that are only 1 to 4 mm in diameter in murine brains that are 10 mm in diameter. This non-invasive imaging method advantageously permits substantially higher resolution imaging than currently available. This improved imaging can be used to image small hemorrhages with higher resolution than current state of the art methods.

The present invention makes use of advanced [¹⁸F]-PET and FL imaging equipment. This equipment offers order-of-magnitude resolution improvements and robotic control over current clinical equipment. Yet, few fluorescent and ¹⁸F-PET tracers are approved for human use. This lack of availability of contrast agents currently limits the use of advanced bioimaging and bioengineering equipment, including the PET/MR scanner and robotic platforms for image-guided surgery.

The present invention provides a general class of [¹⁸F]-PET/FL precursors for small-molecule and peptide drug labeling. The advantages of fusing ¹⁸F-PET and FL chemistries include, for example, reduced time and cost in new contrast development. In a two-for-one strategy, the regulatory approval (safety) of a single molecular reagent will simultaneously clear both PET and FL imaging modalities for in vivo use. In employing reagents with shared [¹⁸F]-PET/FL molecular structures, resulting methodologies for conjugation, radiochemistry, and post-radiolabeling chromatography will be shared. Translational costs will be reduced, specifically GMP synthesis and toxicological assessment (vs. cost of developing independent, stand-alone PET or FL probes).

A combined PET/FL probe is superior to co-injected mixtures of stand-alone PET or FL contrast agents, in that differences in blood clearance, non-specific tissue accumulation, ligand affinity, and receptor saturation do not need to be addressed. The imaging agents described herein are useful in image-guided surgery. PET and FL are additionally useful together, where, for example: (1) PET allows for pre-surgical planning by distinguishing disseminated (oligometastatic disease) from localized cancer; (2) FL allows for intra-operative surgical guidance, where the extent of a resection is clearly demarcated; and (3) FL allows for margin and node confirmation in triplicate i.e., by the surgeon—in vivo observation of unresected margins in the open surgical site and ex vivo in FL/gamma scintillated analysis of resected tissue; and by the pathologist—ex vivo in FL frozen section intraoperative consult. The subject invention improves the efficacy of cancer management by providing persisting, cancer-specific contrast that is useful to multiple specialists on tumor boards (radiologists, urologists, and pathologists) and allows additional FL histology, and FL-assisted cell sorting of resolved cells following surgery. In a cell-imaging embodiment, fluorescence allows post-surgical fluorescence activated cell sorted (FACS) isolation of cells with characteristics that are selected due to assistance from the subject agents.

In a first particular set of embodiments, the method includes administering to the subject an imaging agent comprising the following structure:

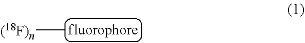

wherein n is an integer of at least 1, and the one or more fluorine-18 (¹⁸F) radionuclides in Formula (1) are bound directly or indirectly to the fluorophore; and (ii) imaging internal biological tissue of said subject by PET or SPECT. For example, in some embodiments, the method is directed to imaging of a lymph node or cerebral spinal fluid (CSF) by administering a composition according to Formula (1) to a subject.

In the case of imaging a lymph node, the method may more specifically employ an imaging agent having the following structure:

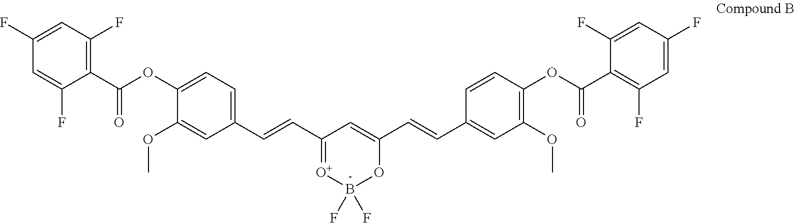

In the case of imaging CSF, the method may more specifically employ an imaging agent having the following structure:

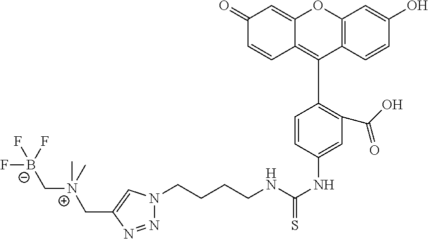 [SMILES: C[N+](C)(Cc1cn(CCCCNC(=S)Nc2ccc(-c3c4ccc(=O)cc-4oc4cc(O)ccc34)c(C(=O)O)c2)nn1)C[B-](F)(F)F]

In a second particular set of embodiments, the method includes administering to the subject an imaging agent comprising the following structure:

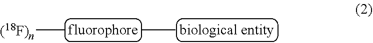

In Formula (2), the biological entity can be any biologically relevant or biologically derived molecule that imparts a further imaging capability, such as by promoting selective targeting of a specific tissue desired to be imaged. For example, in some embodiments, a composition according to Formula (2) is particularly directed to imaging of hemorrhages, in which case the biological entity may be selected as a blood cell. Thus, an imaging agent with particularly useful characteristics in imaging hemorrhages (or another condition where blood flow or pooling plays a role) may have the following structure:

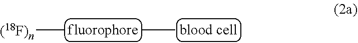

In other embodiments, a composition according to Formula (2) may be particularly directed to imaging of prostate cancer tissue, wherein a PSMA-targeting agent serves as the biological entity. The structure of such an imaging agent may be as follows:

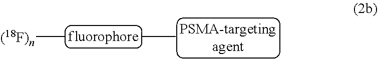

In the case of imaging prostate cancer tissue, the imaging agent may have the following more specific structure:

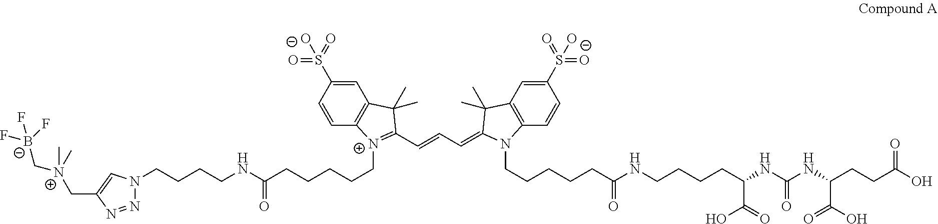

wherein the

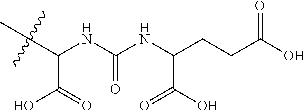 [SMILES: *C(NC(=O)NC(CCC(=O)O)C(=O)O)C(=O)O]

moiety is a PSMA-targeting agent.

### BRIEF DESCRIPTION OF THE FIGURES

FIG. 1 is a schematic process of labeling red blood cells (RBCs) with ¹⁸F-labeled fluorophore compositions 1 and 2 (i.e., RBC-1 and RBC-2), wherein, as shown, composition 1 contains Cy3 as the fluorophore and composition 2 contains Cy5 as the fluorophore.

FIGS. 2A-2C are graphs showing the results for a luminescent ATP detection assay (FIG. 2A), a cell-permeant calcein assay (FIG. 2B), and a cell proliferation assay (FIG. 2C) for RBCs labeled with ¹⁸F-labeled fluorophore compositions 1 and 2 (i.e., RBC-1 and RBC-2, respectively).

FIG. 3 is a graph showing the results of radiochemical analysis of [¹⁸F]-RBC purification by centrifugation under three different labeling conditions: (A) NHS containing composition 1 was labeled with 60 mCi of [¹⁸F]-fluoride ion in a 1 to 4 ratio of composition 1 to fluoride. The mixture was concentrated before neutralization, RBC addition, and PBS washes. The resulting [¹⁸F]-RBC activity obtained was 1.6 mCi after 6 PBS washes; (B) To confirm that RBCs do not take up fluoride nonspecifically, NHS-1 was labeled with 71 mCi of [¹⁸F]-fluoride ion in a 1 to 3 ratio of 1 to fluoride. Concentration of this mixture to a fluoride concentration that is greater than 20 mM was not performed before neutralization and RBC addition. The resulting [¹⁸F]-RBC activity obtained was 4 μCi, 400 fold less (3.5 hour synthesis, decay uncorrected), and could not be imaged in brain in or ex vivo; (C) The labeling of 1 was performed a third time, with 46 mCi of [¹⁸F]-fluoride ion, in the presence of a large excess of ¹⁹F carrier fluoride in a 1 to 125 ratio of 1 to fluoride. Concentration of the mixture was performed before neutralization, RBC addition, and PBS wash. The resulting [¹⁸F]-RBC activity obtained was <1 μCi.

FIG. 4 shows fluorescence images obtained using composition 2 (RBC-2) to image hemorrhage in vivo (i.e., by use of near infrared imaging of RBCs labeled with ¹⁸F-Cy5). Real time observation of traumatic progression is shown. The following images are shown in FIGS. 4: (A) 1 to 5 minute post-lesion, (B) 25 minute post-lesion, and (C) 45 minute post-lesion in a skull-exposed cryolesion bearing mouse (note the growing blood pool), (D) ex vivo fluorescence imaging showing blood pool in the brain, and (E) bright-field imaging confirming site of lesion and hemorrhage. Notably, imaging in (E) is clearer than (A-C) due to removal of skull in (E).

FIGS. 5A and 5B show images resulting from ex vivo RBC-1 [¹⁸F]-PET imaging of intracranial hemorrhage. FIG. 5A shows an ex vivo PET/CT brain image of a tail vein injection of RBC-1 of a mouse 40 minutes after cryolesion. FIG. 5B shows ex vivo bright field imaging after week-long PFA storage.

FIG. 6 shows in vivo [¹⁸F]-PET, brightfield, and [¹⁸F]-PET/CT imaging results for (A) cryolesion initiated ICH, followed by RBC-1 injection 11 minutes later, and (B) cryolesion initiated ICH, followed by RBC-1 injection 25 minutes later.

FIGS. 7A-7G show the scintillated biodistribution of RBC-1 60 minutes after cryolesion and tail vein injection. FIG. 7A shows the general biodistribution of RBC-1 after 50 minutes following tail vein injection to lung, spleen, and liver. FIGS. 7B and 7C show [¹⁸F]-scintillated biodistribution reported in percent injected dose (% ID) (FIG. 7B) and percent injected dose per gram (% ID/g) (FIG. 7C). FIG. 7D shows ex vivo bright-field imaging of brains bearing intracranial hemorrhage in cryolesion cohort. FIG. 7E shows images of brains of mice in control cohort. Notably, the brain hemorrhage did not significantly affect distribution of RBC-1 in other tissues (scale bar: 0.5 cm). FIGS. 7F and 7G show ventral and side PET/CT projections, respectively, confirming distribution data in FIGS. 7B and 7C.

FIG. 8 shows the fraction of viable cells from cell viability studies carried out as described for FIGS. 2A-2C. High-concentration solutions of RBC-1 were incubated with different immortal glial cell lines.

FIG. 9 is a Kaplan-Meier Plot showing that cryolesion/PET associated hypothermia can be avoided with recovery between cryolesion and scanning

FIG. 10A shows the UV-Vis trace at an absorbance at 450 nm (bottom) and radioactivity trace (top) to characterize 6F-Cur-BF₂ radiolabeling. The UV-Vis absorbance at 450 nm was used to monitor 6F-Cur-BF2 elution. Unreacted, contaminating fluoride-18 ion is visible at 1 min in the radiotrace. FIG. 10B shows the radioactivity trace of purified [¹⁸F]-6F-Cur-BF₂ in DMSO, wherein contaminating fluoride-18 ion is removed.

FIG. 11. presents murine data showing that a ¹⁸F-labeled-PSMA-targeted-[¹⁸F]-PET/NIRF (Cy3) contrast agent remains fluorescent long after ¹⁸F has decayed. A mouse is injected with a positron-emitting and fluorescent PSMA-targeted agent. PSMA tissue retains >50% of its maximum fluorescent intensity for at least 14 days post-injection. The mouse had contralateral PSMA− (left flank) and PSMA⁺ (right flank) xenografts. These data are evidence that a patient can receive a single injection of the present PET/Fluorescent agent, have a ¹⁸F PET/CT or PET/MR scan, and then receive a delayed fluorescence-guided radical prostatectomy.

FIGS. 12A and 12B present human Cy3-fluorescent prostate histopathology showing that the present [¹⁸F]-PET/FL agent would be useful regarding human PSMA biopsies. A patient received IV 0.15 mg of PSMA⁺-targeted-[¹⁸F]-PET/NIRF (Cy3) (120 nmol) ˜1 h prior to receiving a standard-of-care prostate cancer biopsy. Tissue was collected, sectioned (frozen), and analyzed by confocal microscopy. FIG. 12A shows Cy3 fluorescence (pink)/DAPI (nuclear stain-blue) histology with pink PSMA⁺ glands. FIG. 12B shows confirmative PSMA⁺ immunohistochemical staining in a neighboring section. Note that PET/FL-Cy3 fluorescence (pink in A) corroborate glandular PSMA⁺ IHC (brown in FIG. 12B). PET/FL-Cy3 fluorescence is only present in abnormal gland bearing PSMA⁺ tissue.

FIGS. 13A-13D provide preliminary data showing that the present [¹⁸F]-PET/FL agent has use in humans. Patient #1 has a case of disseminated PSMA⁺ cancer. FIG. 13A shows that PSMA⁺-targeted-[¹⁸F]-PET/NIRF (Cy3) was ¹⁸F-radiolabeled (FIG. 13B), and injected into the right cephalic vein of Patient #1. FIG. 13C shows that an existing [⁶⁸Ga]-PSMA-11 PET (MIP) scan served as a control for Patient #1 (90 min p.i., Gleeson >8, biopsy-confirmed-PSMA⁺, PET (red) CT (grey)). FIG. 13D shows a PET scan with [¹⁸F]-PET/NIRF (Cy3) performed 8 months later (90 min p.i., 3.6 mCi, <100 μmol) reveals the same major lesions (a-d). Patient #1 received [¹⁷⁷Lu]-PSMA-617 therapy and hip replacement surgery in between the scans shown in FIG. 13C and FIG. 13D. Informed consent/1964 Helsinki declaration/amendment-aligned-institutional approvals were obtained. [¹⁸F]-PET/FL-Cy3 PET shows advanced, disseminated disease, and suggests that Patient #1 is not a candidate for radical prostatectomy.

FIGS. 14A-14F present preliminary data in a second patient showing that the present [¹⁸F]-PET/FL agent has PET use regarding human biopsies and intraoperative radical prostatectomy (back table intraoperative consult). Patient #2 has localized PSMA⁺ cancer and is a candidate for PSMA-targeted-[¹⁸F]-PET/NIRF (Cy3)-guided radical prostatectomy. FIG. 14A Control, A [¹⁸F]-PSMA-1007 PET (MIP) image in a healthy, non-cancerous patient (from EJNMMI; 678) is like the one in FIG. 14B, showing a [¹⁸F]-PET/FL (Cy3) PET (MIP) scan of Patient #2 (5 mCi, MIP, 5 h p.i.). Patient #2 has localized PSMA⁺ prostate cancer that had not disseminated from the prostate (arrow). FIG. 14C is a [⁶⁸Ga]-PSMA-11 scan of Patient #2 which confirmed the PET scan in FIG. 14B revealing localized PSMA⁺ disease and no lymph node involvement. Localized prostate cancer is indicated with an arrow. FIG. 14D shows that the same PET delineation is observed in FIG. 14B (a [⁶⁸Ga]-PSMA-11 scan) and in FIG. 14D (a [¹⁸F]-PET/FL (Cy3) scan performed one month later (D=90 min p.i.). Patient #2 is an ideal candidate for radical prostatectomy. Without undergoing re-injection, Patient #2 underwent radical prostatectomy 24 hours later (after ¹⁸F decay). FIG. 14E shows that resected tissues were fluorescent and PSMA⁺. FIG. 14F shows that post-surgical prostate image co-registration shows prostate cancer in MRI-ADC (panel (a)), PET (panel (b)), and FL (panel (c)). Unfortunately, post-surgical fluorescence imaging reveals that the urologist left a positive (un-resected) margin in the posterior urethra (red arrow), as shown in panel (d) of FIG. 14F

### DETAILED DESCRIPTION OF THE INVENTION

In one aspect, the present disclosure is directed to compositions useful in the imaging of biological tissue by PET or SPECT. The composition contains blood cells that are labeled with (i.e., bound to) a fluorophore that is bound directly or indirectly with one or more fluorine-18 atoms. The resulting PET- or SPECT-imageable blood cells can be described by the following generic structure:

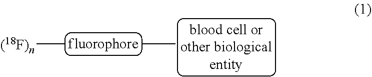

In Formula (1) above, the fluorophore can be any fluorophore acceptable for introduction into a living organism. In some embodiments, the fluorophore is attached to a blood cell, wherein the blood cell can be any type of blood cell, such as a red blood cell (RBC), white blood cell (WBC), or platelet. In the case of a WBC, the WBC can be, more specifically, a granulocyte or agranulocyte, or more particularly, a neutrophil, eosinophil, basophil, lymphocyte, or monocyte. In the case of a lymphocyte, the lymphocyte may be more specifically characterized as a B-cell or T-cell. In other embodiments, the fluorophore is attached to a biological entity other than a blood cell, such as a small molecule, peptide, DNA, aptamer, antibody, or antibody fragment. The variable n is at least 1 (e.g., 1, 2, 3, 4, 5, or more), which indicates the presence of an equivalent number of fluorine-18 atoms. The solid line connecting the fluorophore with the blood cell, as well as the solid line connecting the fluorophore with the one or more fluorine-18 atoms, represent covalent bonds. The two covalent bonds are independently representative of direct or indirect (i.e., via a linker) covalent bonds. In the case of fluorine-18 atoms being bound indirectly to the fluorophore, the one or more fluorine-18 atoms may be bound to a boron or silicon atom, wherein the boron atom or silicon atom is also bound directly or indirectly to the fluorophore. The linker can be or include, for example, an alkylene (—CH₂)ₘ— linker (with m being, for example, 1, 2, 3, 4, 5, or 6) and optionally containing one or more —O— and/or —C(O)— linkages. The linker may also be or include one or more ring linkers, such as phenylene or 1,4-piperazine. The linker may also include a boron or silicon atom on which one or more ¹⁸F atoms reside. In particular embodiments, one or more fluorine-18 radionuclide atoms reside on a (—BF₂—)⁻ linking group, (—BF₃)⁻ terminal group, —SiF₂— linking group, or —SiF₃ terminal group.

As used herein, the term “fluorophore” (or “fluorescing species”) refers to a compound possessing a fluorescent property when appropriately stimulated by electromagnetic radiation. The fluorophores considered herein can absorb and emit light of any suitable wavelength. In some embodiments, it may be desired to select a fluorophore with particular absorption and emission characteristics. For example, in different embodiments, the fluorophore absorbs at nanometer (nm) wavelengths of 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, or 800 nm, or within a range bounded by any two of the foregoing values. In different embodiments, the fluorophore emits at any of the foregoing wavelengths, or within a range bounded by any two of the foregoing values, wherein it is understood that a fluorophore generally emits at a longer wavelength than the absorbed wavelength. The impinging electromagnetic radiation (i.e., which is absorbed by the fluorophore) can be in a dispersed form, or alternatively, in a focused form, such as a laser. Moreover, the absorbed or emitted radiation can be in the form of, for example, far infrared, infrared, far red, visible, near-ultraviolet, or ultraviolet.

The fluorophores considered herein are organic fluorophores, which generally contain at least one carbon-carbon bond and at least one carbon-hydrogen bond. In different embodiments, the organic fluorophore can include, for example, a charged (i.e., ionic) molecule (e.g., sulfonate or ammonium groups), uncharged (i.e., neutral) molecule, saturated molecule, unsaturated molecule, cyclic molecule, bicyclic molecule, tricyclic molecule, polycyclic molecule, acyclic molecule, aromatic molecule, and/or heterocyclic molecule (i.e., by being ring-substituted by one or more heteroatoms selected from, for example, nitrogen, oxygen and sulfur). In the particular case of unsaturated fluorophores, the fluorophore contains one, two, three, or more carbon-carbon and/or carbon-nitrogen double and/or triple bonds. In a particular embodiment, the fluorophore contains at least two (e.g., two, three, four, five, or more) conjugated double bonds (i.e., a polyene linker) aside from any aromatic group that may be in the fluorophore. In other embodiments, the fluorophore is a fused polycyclic aromatic hydrocarbon (PAH) containing at least two, three, four, five, or six rings (e.g., naphthalene, pyrene, anthracene, chrysene, triphenylene, tetracene, azulene, and phenanthrene) wherein the PAH can be optionally ring-substituted or derivatized by one, two, three or more heteroatoms or heteroatom-containing groups. In some embodiments, the fluorophore contains a polyalkyleneoxide group that contains at least two, three, or four alkyleneoxide units. In other embodiments, the fluorophore contains at least one sulfonic acid or sulfonate salt group.

In other embodiments, the organic fluorophore is a xanthene derivative (e.g., fluorescein, rhodamine, Oregon green, eosin, and Texas Red), cyanine or its derivatives or subclasses (e.g., streptocyanines, hemicyanines, closed chain cyanines, phycocyanins, allophycocyanins, indocarbocyanines, oxacarbocyanines, thiacarbocyanines, merocyanins, and phthalocyanines), naphthalene derivatives (e.g., dansyl and prodan derivatives), coumarin and its derivatives, oxadiazole and its derivatives (e.g., pyridyloxazoles, nitrobenzoxadiazoles, and benzoxadiazoles), pyrene and its derivatives, oxazine and its derivatives (e.g., Nile Red, Nile Blue, and cresyl violet), acridine derivatives (e.g., proflavin, acridine orange, and acridine yellow), arylmethine derivatives (e.g., auramine, crystal violet, and malachite green), and tetrapyrrole derivatives (e.g., porphyrins and bilirubins).

In some embodiments, the fluorophore is a streptocyanine (open chain cyanine) having the general structure:

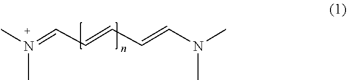

wherein n in formula (1) above can be, for example, precisely, at least, or no more than 0, 1, 2, 3, 4, 5, 6, 7, 8, or within a range therein. Other structures related to or derived from formula (1) are also considered herein, as amply described in Guieu, V., et al., Eur. J. Org. Chem., 2007, 804-810, which is incorporated herein by reference in its entirety.

In other embodiments, the fluorophore is a hemicyanine having the general structure:

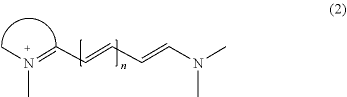

wherein n in formula (2) is as defined above. The arc in Formula (2) indicates a nitrogen-containing ring, such as pyrrolyl. The arc may alternatively represent a bicyclic ring system, such as a benzopyrrolyl fused ring system. Other structures related to or derived from formula (2) are also considered herein, as amply described in Stathatos, E., et al. Chem. Mater., 2001, 13, 3888-3892, and Yao, Q.-H., et al. J. Mater. Chem., 2003, 13, 1048-1053, which are incorporated herein by reference in their entirety.

In other embodiments, the fluorophore is a closed cyanine having the general structure:

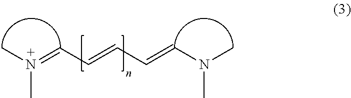

wherein n in formula (3) is as defined above.

In particular embodiments, the fluorophore is a cyanine dye (i.e., cyanine-based fluorophore). The term “cyanine dye”, as used herein, refers to any of the dyes, known in the art, that include two indolyl or benzoxazole ring systems interconnected by a conjugated polyene linker. The cyanine dye typically contains at least two or three conjugated carbon-carbon double bonds, at least one of which is not in a ring, such as depicted in any of Formulas (1)-(3). The cyanine dye (or other type of dye) often contains at least two pyrrolyl rings. Some particular examples of cyanine dyes are the Cy® family of dyes, which include, for example, Cy2, Cy3, Cy3B, Cy3.5, CyS, Cy5.5, Cy7, and Cy9. The term “cyanine moiety”, as used herein, generally includes the bis-indolyl-polyene or bis-benzoxazolyl-polyene system, but excludes groups attached to the ring nitrogen atoms in the indolyl or benzoxazolyl groups. The cyanine dyes may also include the Alexa® family of dyes (e.g., Alexa Fluor 350, 405, 430, 488, 500, 514, 532, 546, 555, 568, 594, 610, 633, 647, 660, 680, 700, 750, and 790), the ATTO® family of dyes (e.g., ATTO 390, 425, 465, 488, 495, 520, 532, 550, 565, 590, 594, 601, 615, 619, 629, 635, 645, 663, 680, 700, 729, and 740), and the Dy® family of dyes (e.g., DY 530, 547, 548, 549, 550, 554, 556, 560, 590, 610, 615, 630, 631, 631, 632, 633, 634, 635, 636, 647, 648, 649, 650, 651, 652, 675, 676, 677, 680, 681, 682, 700, 701, 730, 731, 732, 734, 750, 751, 752, 776, 780, 781, 782, and 831). The ATTO dyes, in particular, can have several structural motifs, including, coumarin-based, rhodamine-based, carbopyronin-based, and oxazine-based structural motifs.

In another aspect, the invention is directed to methods for synthesizing fluorophore compositions of Formula (1). The method generally involves the following reaction scheme:

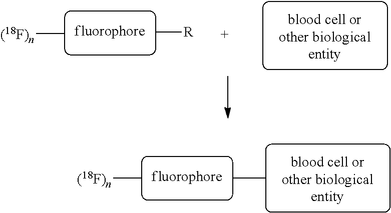

In the above scheme, the “R” group represents a reactive crosslinking group capable of binding to the blood cell. The term “reactive crosslinking group”, as used herein, is any group that can interact or react with chemical groups present on the blood cell such that the fluorophore becomes permanently affixed or attached to the blood cell (i.e., with no detachment of the fluorophore from the blood cell). Some examples of reactive groups include amino-reactive, carboxy-reactive, thiol-reactive, alcohol-reactive, phenol-reactive, aldehyde-reactive, and ketone-reactive groups. Some examples of amino-reactive groups include carboxy groups (—COOR′, where R′ is H or hydrocarbon group), activated ester groups (—COOR′, where R′ is a carboxy-activating group, such as deprotonated N-hydroxysuccinimide, i.e., NHS), carbodiimide ester groups (e.g., EDC), tetrafluorophenyl esters, dichlorophenol esters, epoxy (e.g., glycidyl) groups, isothiocyanate, sulfonylchloride, dichlorotriazines, aryl halides, and azide, and sulfo-derivatives thereof, and combinations thereof. Some examples of carboxy-reactive groups include amino groups and hydroxyalkyl groups, typically in the presence of a carboxy group activator to form an activated ester. Some examples of thiol-reactive groups include maleimido (“Mal”) groups, haloacetamide (e.g., iodoacetamide) groups, disulfide groups, thiosulfate, and acryloyl groups. Some examples of alcohol-reactive and phenol-reactive groups include aldehydes, ketones, haloalkyl, isocyanate, and epoxy (e.g., glycidyl) groups. Some examples of aldehyde-reactive and ketone-reactive groups include phenol, hydrazide, semicarbazide, carbohydrazide, and hydroxylamine groups. Other reactive groups include 6-oxyguanine groups and phosphoramidite groups. The term “reactive group” can further encompass any larger group (e.g., a hydrocarbon group, such as a cyclic or aromatic hydrocarbon) on which the reactive crosslinking group is attached. For example, a 6-oxyguanine group may include a ring-containing linking moiety attached to the 6-oxy atom for attaching to the linking portion in Formula (1). In other embodiments, the reactive group may be derivatized, such as by including any of the hydrophilic groups described above, such as sulfonate (e.g., a sulfo-NHS group), carboxy, hydroxy, or halide groups.

The reactive group can also be a group that selectively targets (i.e., binds to and/or reacts with) another molecule that has been conjugated to the blood cell. In particular embodiments, the selective targeting group is a group that can engage in an affinity bond. Some examples of reactive groups that can engage in an affinity bond are biotin (which forms an affinity bond with avidin or streptavidin); avidin or streptavidin (which forms an affinity bond with a biotin molecule); an antibody or fragment thereof that can specifically bind to a molecule bearing an epitope reactive with the antibody; a peptide, oligopeptide, or lectin that can specifically bind to another biomolecule; or a nucleic acid, nucleoside, nucleotide, oligonucleotide, or nucleic acid (DNA or RNA strand) or vector that specifically binds to a complimentary strand.

Some examples of ¹⁸F-fluorophore-R precursors (reactive with blood cells) are shown below:

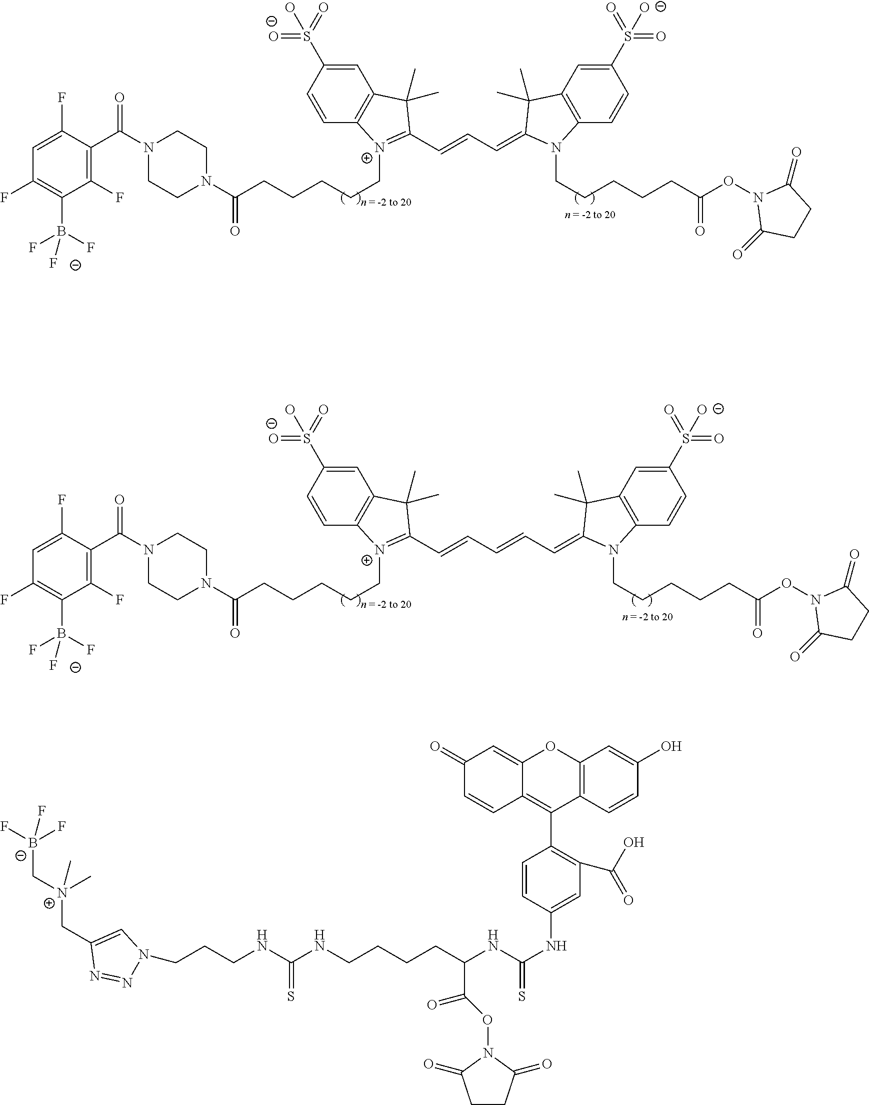

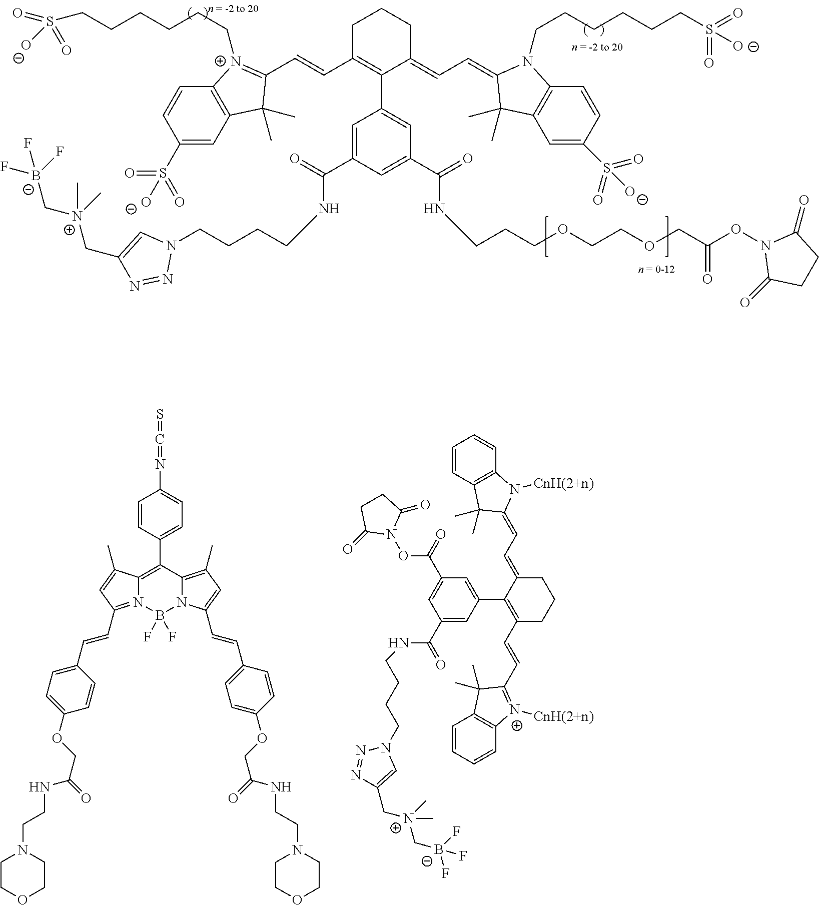

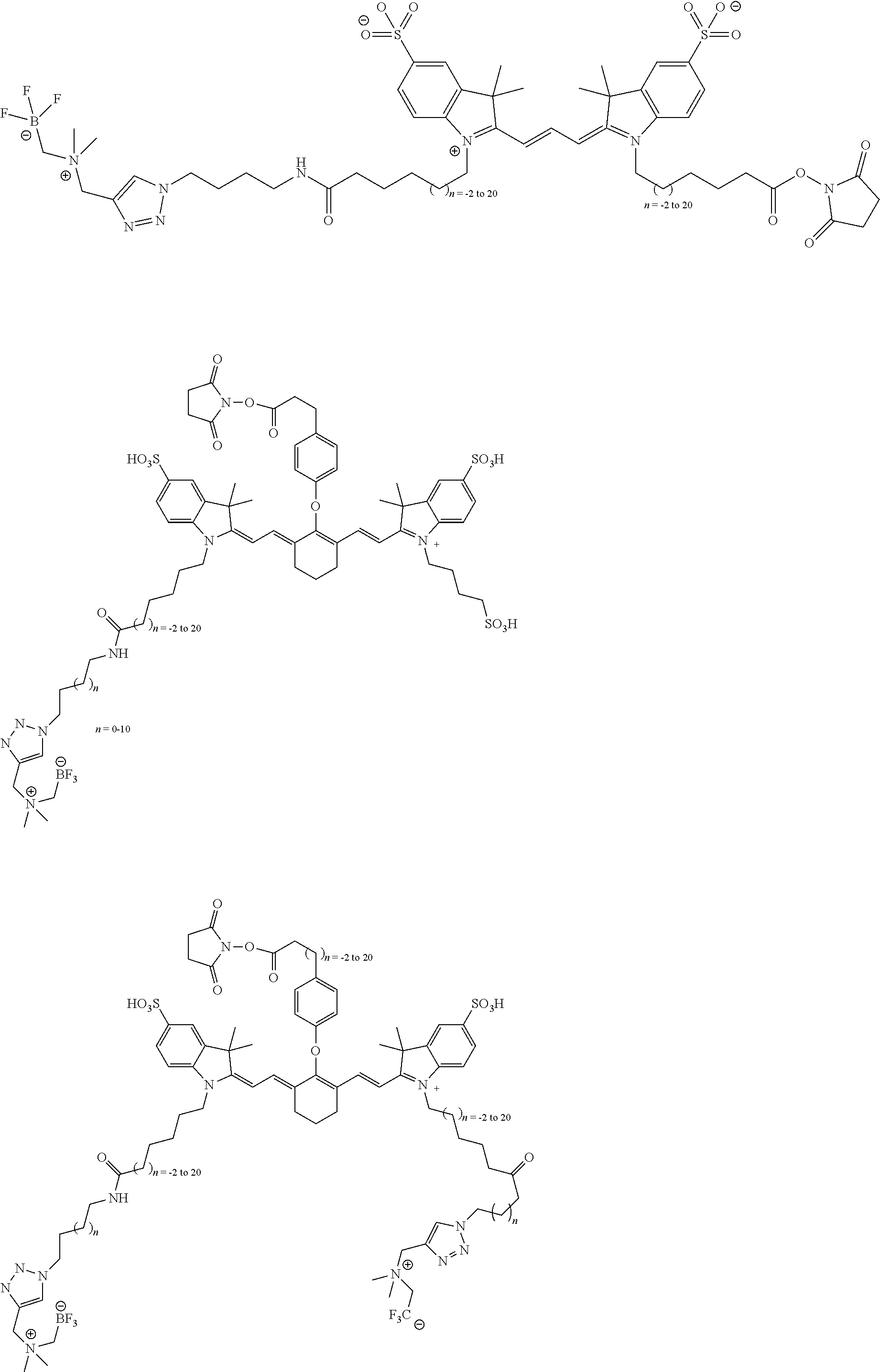

In any the above structures containing an N-hydroxysuccimide (NHS) group, the NHS group may be replaced with, for example, an alkyl, aryl (e.g., phenyl), PEG, amide, ester, or other reactive or non-reactive group.

In a first particular embodiment, the imaging agent contains or consists of (i) a tissue-targeting moiety operatively affixed to (ii) a fluorophore. In a second particular embodiment, the imaging agent contains or consists of (i) a fluorine atom-containing moiety operatively affixed to (ii) a fluorophore, which in turn is operatively affixed to (iii) a tissue-targeting moiety. In a third particular embodiment, the imaging agent contains or consists of (i) a fluorine atom-containing moiety operatively affixed to (ii) a fluorophore which, by virtue of its chemical nature, permits visualizing a tissue of interest.

A tissue-targeting moiety ensures that the imaging agent permits visualization of the tissue of interest by specifically interacting with that tissue (e.g., by adhering to that tissue). Tissue-targeting moieties include, without limitation, an agent that specifically binds to PSMA (e.g., a PSMA inhibitor, such as “Compound A” depicted earlier above), and a blood cell (e.g., RBC).

A fluorophore permits visualization of the tissue of interest by, for example, fluorescence imaging and “optical” imaging (such as visual observation with the naked eye). Fluorophores include, for example, cy3, cy7, fluorescein, and any of the fluorophores known in the art, such as those described above. In some embodiments, the fluorophore is preferably a cyanine fluorophore, and more particularly, a hydrophilic cyanine fluorophore.

A fluorine atom-containing moiety permits visualization of the tissue of interest by, for example, PET imaging. Fluorine atom-containing moieties include, without limitation, fluorine captors, such as those described above. Preferably, the moiety contains either two or three fluorine atoms, which can be either ¹⁸F or ¹⁹F (“^{18/19}F”). Preferred agents for this invention include, without limitation, (i) an ^{18/19}F-containing moiety (either two or three ^{18/19}F atoms) operatively affixed to (ii) a cy3 fluorophore, which in turn is operatively affixed to (iii) a PSMA-targeting moiety. As a specific example, this invention makes use of “Compound A,” as described above, for this purpose. Another preferred agent for purposes of the invention is the following compound (“Compound B”). Both the ¹⁸F-containing and ¹⁹F-containing embodiments of Compounds A and B are considered herein. The structures of Compounds A and B are provided as follows:

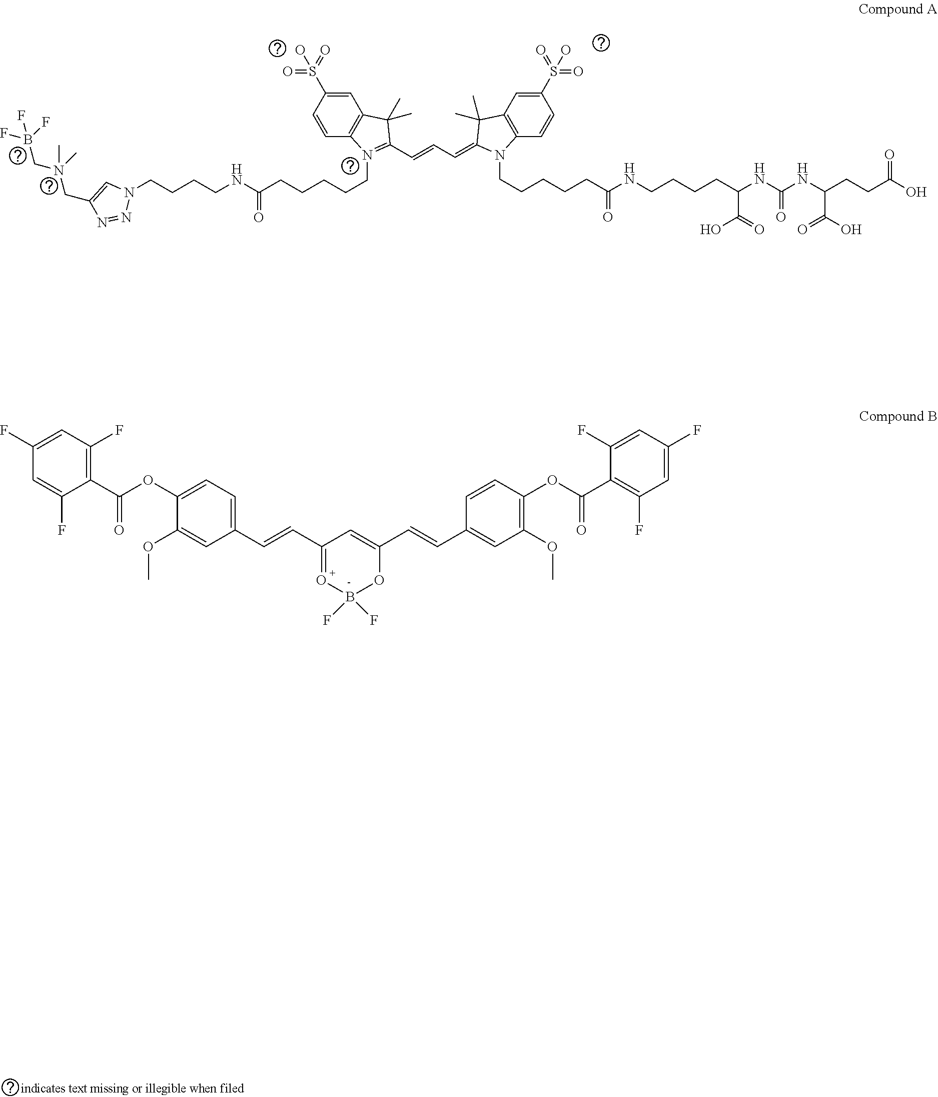

In another embodiment, the imaging agents target Fibroblast Activation Protein, HER2, CXCR4, or another biomarker, such as any of those described in O'Connor et al., Nature Reviews, vol. 14, pp. 169-186, March 2017, the contents of which are herein incorporated by reference in their entirety. The subject agents include, without limitation, small molecules, DNAs, aptamers, antibodies, and antibody fragments.

The ¹⁸F-fluorophore-R molecule can be prepared by, for example, functionalizing a fluorophore with R reactive groups and a labile fluorine-containing group (e.g., —BF₃⁻ or —SiF₃) to produce a ¹⁹F-fluorophore-R molecule, and then contacting the ¹⁹F-fluorophore-R molecule with aqueous H[¹⁸F] under conditions (e.g., acidic pH, such as ˜2.5) where the ¹⁸F isotopically exchanges with ¹⁹F atoms, thereby resulting in at least one ¹⁸F per boron or silicon atom. The isotopic exchange method is described in, for example, U.S. Pat. No. 8,114,381, the contents of which are herein incorporated by reference.

In another aspect, the present disclosure is directed to pharmaceutical compositions containing the above-described ¹⁸F-fluorophore-blood compositions. Typically, in order for the ¹⁸F-fluorophore-blood composition to be administered to a subject, the fluorophore composition is formulated together with one or more pharmaceutically acceptable carriers (additives) and/or diluents, as well known in the art of pharmaceutical compositions. For purposes of the invention, the labeled blood composition is typically formulated as a liquid for administration by injection. The phrase “pharmaceutically acceptable” is used herein to refer to those compounds, materials, compositions, and/or dosage forms that are, within the scope of sound medical judgment, suitable for entering a living organism or living biological tissue, preferably without significant toxicity, irritation, or allergic response. In the pharmaceutical composition, the compound is generally dispersed in the physiologically acceptable carrier, by being dissolved or emulsified in a liquid carrier. The carrier should be compatible with the other ingredients of the formulation and physiologically safe to the subject. Examples of suitable aqueous and non-aqueous carriers that may be employed in the pharmaceutical compositions of the invention include, for example, water, ethanol, polyols (such as glycerol, propylene glycol, polyethylene glycol, and the like), vegetable oils (such as olive oil), and injectable organic esters (such as ethyl oleate), and suitable mixtures thereof.

In another aspect, the invention is directed to methods of imaging biological tissue in a subject by administering to a subject any of the above described imaging compositions in which blood cells are labeled with (i.e., bound to) a fluorophore bound directly or indirectly with one or more fluorine-18 atoms. The methods of this invention include, without limitation, those that comprise PET imaging, fluorescence imaging, and fluorescence-based optical imaging. As used herein, the term “administer”, refers to any means to deliver the agent to a subject's body via any known method. Specific methods of administration include, without limitation, intravenous, oral, intramuscular, subcutaneous, and intra-tumoral administration. Typically, any of the imaging (¹⁸F-fluorophore-blood) compositions described above, such as according to Formula (1), is injected into the blood stream (i.e., intravenously) or directly into tissue to be imaged. In some embodiments, the imaging composition described above is further bound to a selective targeting agent, such as a tumor targeting agent, to permit enhanced analysis of the targeted tissue by PET or SPECT in combination with clinical fluorescence imaging. Methods of imaging biological tissue with PET or SPECT is well known in the art, such as described in, for example, P. Zanzonico, Seminars in Nuclear Medicine, vol. XXXIV, No. 2, pp. 87-111, April 2004; G. Mariani et al., Eur. J. Nucl. Med. Mol. Imaging, DOI 10.1007/s00259-010-1390-8, February 2010; and A. Rahmim et al., Nucl. Med. Commun., 29:193-207, 2008, the contents of which are herein incorporated by reference in their entirety.

The imaging method described herein can be used to image cerebral blood flow and general blood pool. The imaging can be used to, for example, assess or monitor the progression of a hemorrhage and for imaging the response to intervention. The hemorrhage may be located in any part of the body, including the brain (e.g., intracerebral hemorrhage, or hemorrhagic or ischemic stroke). Such assessment and monitoring may be especially important for patients with challenged renal function, where MRA and CTA are contraindicated. The imaging method can also be used simultaneously with CT imaging on a PET/CT. The imaging method may also involve PET/MRI development due to the superior images of brain tissue provided by MRI. The imaging method may also be used in intraoperative mode where PET could be used to guide a surgeon to a fluorescent probe in surgical repair. This is especially true in neurosurgery and otolaryngology, where endoscopic cameras, that can be easily adapted to fluorescent procedures, are in regular use. The imaging method may also be based on other cells, which could be used to, for example, track stem cells, T cells, or circulating cancer cells. The method described herein may also be applied to the imaging of traumatic brain injury, intestinal bleeding, renal bleeding, and internal bleeding in emergency situations, wherein the term “bleeding” may be synonymous with “hemorrhaging”. The imaging method may also be used to assess or monitor transplant rejection or acceptance, such as for allotransplants, or more specifically, to image deep tissue kidney allotransplants. The imaging method may also be used to image perfusion, including thrombosis, such as red blood cell perfusion in vascularized composite allotransplantation (VCA). Notably, changes in blood flow are the earliest indicators of VCA complication. As the imaging method can detect changes in blood flow, the imaging method can detect complications in VCA and other transplants. The imaging method may also be used predict vascular thrombosis and indicate regions of necrosis.

The imaging method may include simultaneous imaging of internal biological tissue by fluorescence imaging, wherein fluorescence imaging of biological tissue is well known in the art (e.g., F. Leblond et al., Journal of Photochemistry and Photobiology B: Biology, vol. 98 (1), 77-94, January 2010). In particular, the imaging method can be used to image early vascular thrombosis, such as in reconstructive microsurgery, by fluorescence, and deep tissue VCA by PET. In the fluorescence mode, the imaging (¹⁸F-fluorophore-blood) composition can be used to monitor clinical graft viability and perfusion at high resolution, superficially (in free flaps) or in open surgical sites. Fluorescence imaging can indicate early rejection at the single cell level in superficial transplants (FL). Blood cells are optionally radiolabeled with fluorine-18 to generate a species that is molecularly (electronically) identical to the fluorescent probe. PET technology can be used to make VCA perfusion visible on PET/CT or PET/MRI devices in deep tissue transplants. The imaging (¹⁸F-fluorophore-blood) composition can be used to generate PET profiles of acute failure so that imminent graft failure could be predicted, and allotransplants can be preserved through prompt intervention. The fluorophore compositions described herein can thus prolong transplant lifetime and prevent tissue rejection in transplants. Moreover, patients that receive VCAs generally already have IV catheters in place (for analgesic delivery), thus making IV delivery of labeled blood cells a non-invasive technology.

PET/FL labeled blood cells improve upon directly injected small-molecules (ICG or fluorescein) by not staining the endothelium of vessels, a flaw with quantitative fluorimetry and other small-molecule-dye VCA monitoring technologies. Fluorescence imaging technologies are superior to implantable and surface microwave frequency doppler, as optical technology (300-1000 THz) allows dynamic visualization of single blood cells flowing through arteries and veins. This resolution cannot be achieved with medical doppler imaging (microwave frequency, 3-10 MHz, due to the Abbe diffraction limit). In the PET mode, ¹⁸F-PET-labeled blood cells are earlier indicators of inadequate perfusion vs. competitive PET probes that sense changes to metabolism ([¹⁸F]-FDG) and oxygen supply ([¹⁸F]-hypoxia), changes that come after changes in blood flow. Both PET and fluorescent aspects of this technology make it superior to current technologies for detecting early VCA complication.

In some embodiments, the imaging method is used to assess or monitor the extent or progression of a cancer or pre-cancer, such as by imaging a tumor or pre-cancerous tissue. The cancerous or pre-cancerous tissue being imaged may be located in any part of the body, such as, for example, the prostate, breast (including triple negative breast cancer), brain, lungs, stomach, intestines, colon, rectum, ovaries, cervix, pancreas, kidney, liver, skin, lymphs, bones, bladder, or uterus. The cancer can also include the presence of one or more carcinomas, sarcomas, lymphomas, blastomas, or teratomas (germ cell tumors).

In particular embodiments, tissues to be visualized include, without limitation, any PSMA⁺ tissue regardless of location in the body, and preferably prostate tumor tissue. Methods employing the subject imaging agents include, without limitation, the following:

(i) imaging a PSMA⁺ tumor (e.g., a brain tumor or a prostate tumor) via PET imaging, fluorescence imaging and/or optical imaging;

(ii) performing an (optionally ultrasound-guided) agent-targeted tissue biopsy via PET imaging, fluorescence imaging, and/or optical imaging;

(iii) performing a (optionally ultrasound-guided) surgical procedure on a tumor (e.g., removing a PSMA⁺ tumor, such as a prostate tumor) while employing PET imaging, fluorescence imaging, and/or optical imaging;

(iv) performing a pathology and/or histology analysis of a tissue sample (e.g., a PSMA⁺ tumor sample, such as a prostate tumor sample) via PET imaging, fluorescence imaging, and/or optical imaging (which analysis can comprise, for example, a tumor margin analysis);

(v) determining the status (e.g., size, location and/or stage) of a tumor (e.g., a PSMA⁺ tumor, such as a prostate tumor) via PET imaging, fluorescence imaging, and/or optical imaging;

(vi) monitoring the progress of cancer therapy (e.g., the pharmaceutical treatment of a PSMA⁺ tumor-afflicted subject, such as a subject afflicted with a prostate tumor) via PET imaging, fluorescence imaging, and/or optical imaging; and

(vii) monitoring the progress of cancer surgery (e.g., the surgical removal of a PSMA⁺ tumor, such as a prostate tumor) via PET imaging, fluorescence imaging, and/or optical imaging (including pre-op monitoring, post-op monitoring, and monitoring during surgery).

In some embodiments where an imaging agent, such as Compound A, is employed in the method, the following human dosing is envisioned. Compound A [¹⁸F] at an activity ranging from 3 to 10 mCi, at a mass of 60 to 100 umol (80 to 130 μg) is injected intravenously at least 45 minutes prior to PET scanning Compound A [¹⁸F] is visible in a PET scanner for 0 min to 8 hours post-injection. Co-injected, residual Compound A [¹⁹F] is visible for up to 2 weeks post injection. Compound A fluorescence is visible in PSMA⁺ cancer as early as 45 minutes post-injection. However, it is recommended that fluorescence-guided surgery using Compound A be carried out 24 hours post-injection to allow for quantitative clearance of the agent from the urinary system (bladder) to minimize non-cancer-specific fluorescent signal during surgery.

In some embodiments where an imaging agent, such as Compound B, is employed in the method, the following human dosing is envisioned. Compound B [¹⁸F] at an activity ranging from 3 to 10 mCi, and mass that is less than 100 umol (˜100 μg), is injected intra-tumorally or in relavant fatty tissue prior-to or during a PET scan. Co-injected, residual Compound B [¹⁹F] is visible under the PET scanner from 0 min to 8 hours post-injection and can be used to visualize dynamic flow. Co-injected or residual Compound B [¹⁸F] is visible in the lymph nodes for at least 24 hours post-injection.

This invention further provides an imaging agent (referred to herein as “stable imaging agent” or “stable agent”) comprising a fluorphore affixed to a biological entity, as those two moieties are defined herein with respect to the subject ¹⁸F-containing imaging agents. In one embodiment, the fluorophore comprises ¹⁹F. In a preferred embodiment, the biological entity is a PSMA-targeting agent. In another preferred embodiment, the fluorophore is a Cy3 cyanine dye. In yet another preferred embodiment, the present stable imaging agent has the structure of Compound A or Compound B as described herein, except that each ¹⁸F atom therein is substituted with a ¹⁹F atom.

The immediate chemical precursor to each present ¹⁸F-labeled agent (which is positron-emitting and fluorescent) is an electronically identical agent that contains ¹⁹F in lieu of ¹⁸F. This ¹⁹F analogue is stable. A ¹⁹F-labeled agent is fluorescent but is not useful for positron emission tomography because ¹⁹F is stable and cannot undergo nuclear fission to emit a positron—a requirement for positron emission tomography. The present stable agents can be stockpiled for years at a time, transported, and used as stand-alone fluorescent-only agents, without radioactive hazard. Once a stable agent undergoes ¹⁸F-¹⁹F isotopic exchange, it becomes radioactive, becomes useful for PET, and adopts a shelf life limited by the rapid nuclear decay of ¹⁸F (t½=108 min).

This invention still further provides a method for imaging a subject's biopsy sample (preferably that of a human subject), comprising: (i) contacting the present stable imaging agent with the biopsy sample ex vivo, or (ii) intravenously administering the present ¹⁹F-labeled stable agent or present ¹⁸F-labeled agent to a patient; and imaging the biopsy sample by fluorescence. In a preferred embodiment, the biopsy sample is a core biopsy sample, and the imaging may or may not be guided by transrectal ultrasound, by multiparametric MRI and PET/CT or PET/MR scans produced with [¹⁸F]-positron-emitting and/or fluorescent agent, or by any other form of imaging.

This invention further provides a method for performing surgery on a subject (preferably a human) comprising: (i) contacting the present stable imaging agent with the subject's tissue ex vivo or in situ; or (ii) intravenously administering the present ¹⁹F-labeled stable agent or present ¹⁸F-labeled agent to a patient. In this method, the agent's biological entity targets specific tissue and permits its fluorescent visualization ex vivo or in situ, or while performing surgery on the tissue, wherein the surgery is guided by fluorescence emitted by the tissue to which the agent is bound. In a preferred embodiment, the surgery is guided robotic surgery (e.g., fluorescence-guided robotic surgery). In another preferred embodiment, the surgery is prostate surgery and the biological entity is a PSMA-targeting agent.

This invention still further provides a method for imaging a histopathology sample from a subject (preferably a human), comprising contacting the present stable imaging agent with the sample, and imaging the sample by fluorescence. In a preferred embodiment, the sample is a frozen section, and is imaged intra-operatively for positive margin determination.

This invention further provides a method for visualizing an individual cell (preferably among a group of cells, and preferably ex vivo), comprising contacting the cell (or cell population) with the present stable imaging agent, wherein the agent's biological entity targets the individual cell and permits its fluorescent visualization. In a preferred embodiment, the individual cell is a prostate cell and the present stable agent's biological entity is a PSMA-targeting agent. As an example, the present stable agent can be used to visualize individual PSMA⁺ cells under the microscope, whereby the stable agent guides the selection of specific cancer cells for genetic or epigenetic sequencing (useful, for example, for identifying adjuvant treatments that may improve patient outcome).

The non-fluorine atom portions of the present stable imaging agents are envisioned, mutatis mutandis, as they are for the non-fluorine atom portions of the ¹⁸F-containing imaging agents of this invention. Also, the methods envisioned for using the present stable agents include, without limitation, the following (performed in a manner appropriate for a non-¹⁸F-labeled agent): (i) performing a surgical procedure on a tumor (e.g., removing a PSMA⁺ tumor, such as a prostate tumor) while employing fluorescence imaging and/or optical imaging; (ii) performing a pathology and/or histology analysis of a tissue sample (e.g., a PSMA⁺ tumor sample, such as a prostate tumor sample) via fluorescence imaging and/or optical imaging (which analysis can comprise, for example, a tumor margin analysis); (iii) determining the status (e.g., size, location and/or stage) of a tumor (e.g., a PSMA⁺ tumor, such as a prostate tumor) via fluorescence imaging and/or optical imaging; (iv) monitoring the progress of cancer therapy (e.g., the pharmaceutical treatment of a PSMA⁺ tumor-afflicted subject, such as a subject afflicted with a prostate tumor) via fluorescence imaging and/or optical imaging; and (v) monitoring the progress of cancer surgery (e.g., the surgical removal of a PSMA⁺ tumor, such as a prostate tumor) via fluorescence imaging and/or optical imaging.

In another aspect, the present invention is directed to a kit for making and/or using any of the above-described imaging agents. The kit may include, for example, a ¹⁹F-bearing or boronic ester fluorescent precursor as a targeted biological agent or a NHS ester for general reaction. The kit may include instructions for mixing aliquots of these compositions with ¹⁸F-containing acidic water to give the ¹⁸F-bearing PET-visible composition. The kit may also include a commercial column for passing the composition through to remove contaminating fluoride ion prior to patient administration (e.g., via injection).

Examples of kit-based preparations include protocols described in the following: (i) (preparation from a boronic ester)—Wang, Y., An, F., Chan, M., Friedman, B., Rodriguez, E. A., Tsien, R. Y., Aras, O., and Ting, R. (2017) “18F-positron-emitting/fluorescent labeled erythrocytes allow imaging of internal hemorrhage in a murine intracranial hemorrhage model.” J. Cerebral Blood Flow and Metabolism., 37(3), 776-786. PMID: 28054494; (ii) (preparation from a 19F-bearing molecule)—Kommidi, H., Guo, H. Nurili, F., Vedvyas, Y., Jin, M. M., McClure, T., D., Ehdaie, B., Sayman, H., Akin O., Aras, O., Ting, R. (2018) “18F-positron emitting/trimethine cyanine-fluorescent contrast for image-guided prostate cancer management.” J. Med. Chem. 61, 4256-4262; and (iii) Kommidi, H., Guo, H., Chen, N., Kim, D., He, B., Wu, A. P., Aras, O, Ting, R. (2017) “A [18F]-positron-emitting, fluorescent, cerebrospinal fluid probe for imaging damage to the brain and spine.” Theranostics. 7, 2377-2391. (Cover article) PMID: 28744321, wherein the contents of references (i)-(iii) are herein incorporated by reference in their entirety.

In a preferred embodiment, a Compound A kit comprises dry [¹⁹F]-Compound A along with solutions for preparation, including (i) an acidic solution for radiolabeling (i.e. pH=2.0, pyridazine-HCl buffer, ˜10 μL) (this could be any acid, such as hydrochloric acid), and a C18 cartridge (e.g., Waters No. 186005125) for the user to purify their labeled agent. Additional optionally included solutions include one for purification (e.g., a 20-23 mL volume of water to flush contaminating [¹⁸F]-fluoride ion from [¹⁸F]-Compound A that is bound on the cartridge), a 4.0 mM HCl solution in ethanol (99%) (to elute [¹⁸F]-Compound A after removal of [¹⁸F]-fluoride ion), and 1 mM phosphate buffered saline (1× PBS) to neutralize [¹⁸F]-Compound A. The kit also optionally contains a 0.22 μm filter for the agent to be administered (e.g., injected) to the patient. The user will have to provide their own ¹⁸F-fluoride ion from a cyclotron. All solutions are sterile. Optionally, the kit includes only [¹⁹F]-Compound A and a C18 cartridge (e.g., Waters No. 186005125), and users can make their choice of washing solutions.

In a preferred embodiment, a Compound B kit comprises (i) dry Compound B, (ii) a solution of tin(IV) chloride, and (iii) HPLC grade, dry acetonitrile. The user would provide their own ¹⁸F-fluoride ion from a cyclotron. After drying this ¹⁸F-fluoride, the users would mix all reagents. For Compound B, no purification cartridge is needed (although one could use a cartridge). The user would simply precipitate out Compound B with water, wash a few times with water to remove all fluoride ion, then re-suspend Compound B in a PBS-buffered DMSO solution that would be passed over a 0.22 μpm filter for Compound B to be injected, e.g., intratumorally.

Examples have been set forth below for the purpose of illustration and to describe the best mode of the invention at the present time. However, the scope of this invention is not to be in any way limited by the examples set forth herein.

### EXAMPLES

Synthesis of tri- and penta-methylene N-hydroxy succinimide bearing boronate fluorescent probes (1) and (2)

The synthesis of the PET/NIRF agents, 1 and 2, was done in a single, two-step reaction. The final yield of 1 was 30%. The final yield of 2 was 15%.

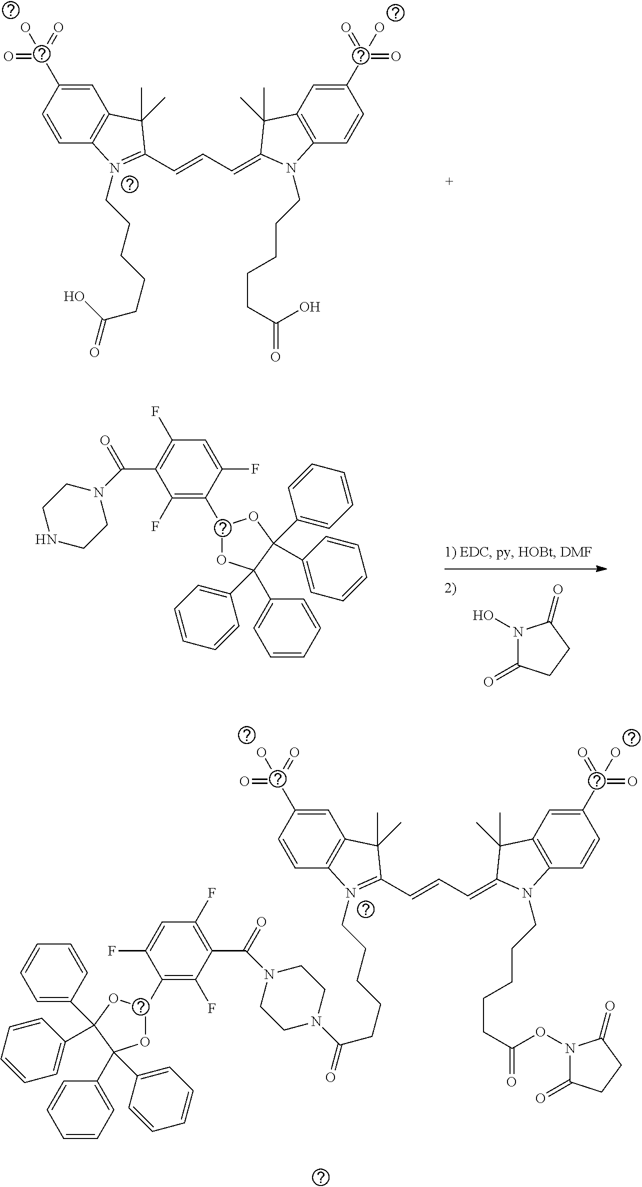

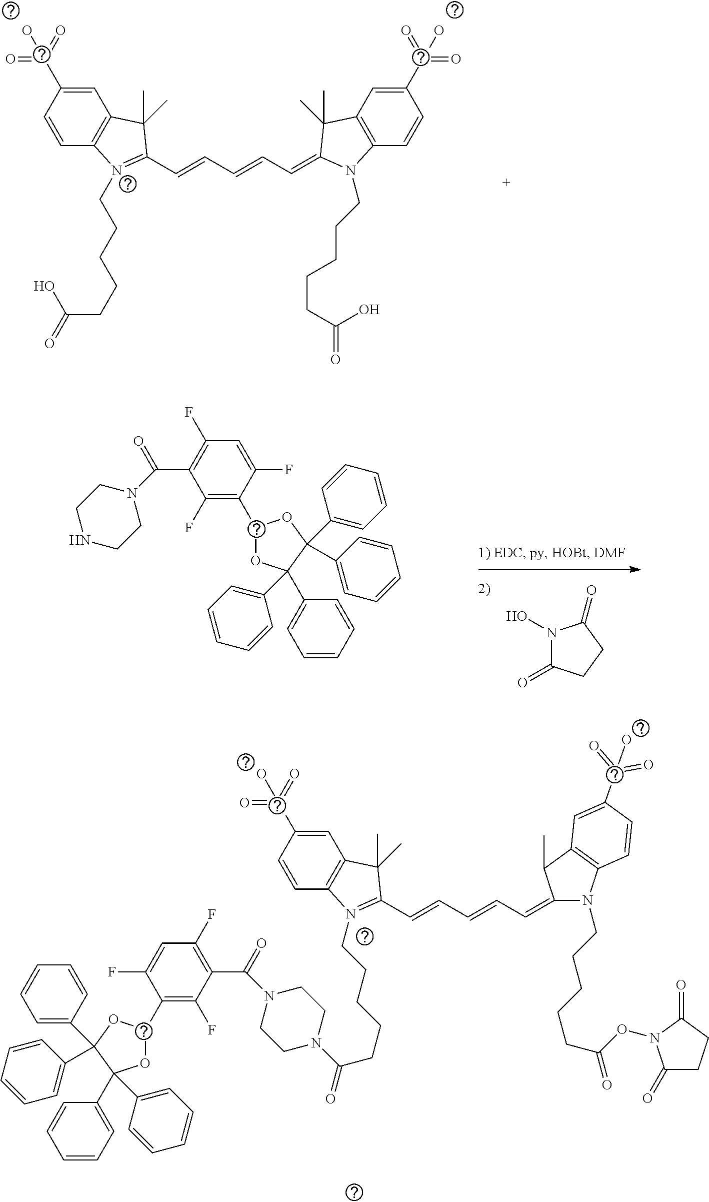

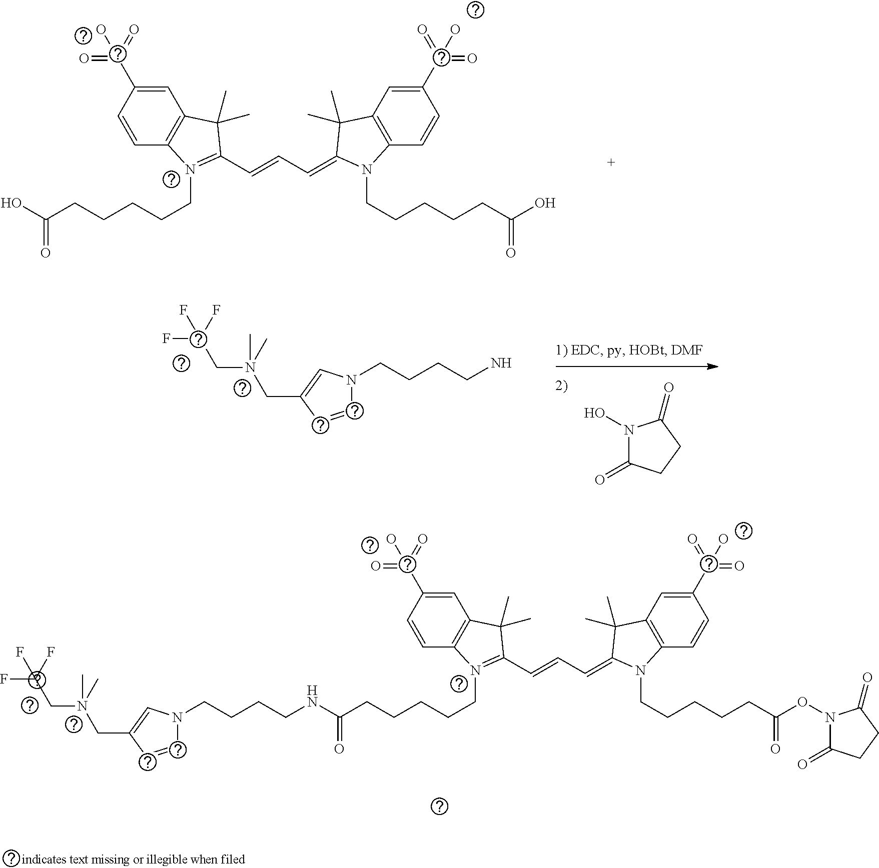

Synthesis of dioxaborolane bearing trimethine cyanine modified NHS ester (1)

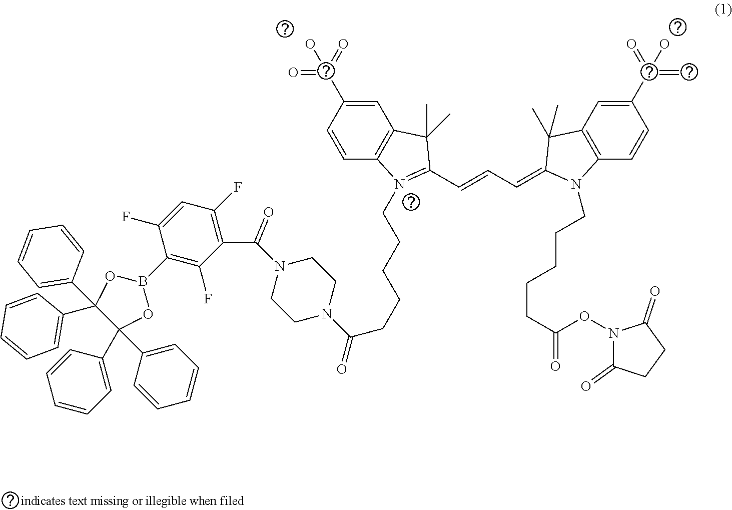

The following reagents were added to a 1.3 mL glass v-vial in the following order: CY3.18.OH (12 mg, 17 μmols), Piperazin-1-yl(2,4,6-trifluoro-3-(4,4,5,5-tetraphenyl-1,3,2-dioxaborolan-2-yl)phenyl)methanone (12 mg, 19 μmols), hydroxybenzotriazole monohydrate (HOBt, 14 mg, 91 μmols), 800 μL dimethylformamide (DMF), 80 μL pyridine, and N-(3- dimethylaminopropyl)-N′-ethylcarbodiimide hydrochloride (EDC, 150 mg, 785 μmols). The reaction proceeded for 2.5 hours at room temperature before an excess of N-hydroxy succinimide (15 mg, 130 μmols) was added. The reaction was left overnight at room temperature before it was chromatographed by preparative, reverse phase HPLC. Fractions containing 1 were lyophilized to give 1 (7.2 mg, 5.1 μmols, pink solid). The resulting solid was dissolved in DMSO and distributed into 1 mM 20 μL aliquots which are stored at −78° C. UPLC-MS+: a10-90%. H2O: ACN (0.05% TFA), 1.5 min gradient, 0.6 mL/min flow, det. 1414 M/Z, 550, 280, 215 nm Abs spectra. Elution time: 1.71 min HRMS (ESI) calculated for C₇₆H₇₆BF₃N₅O₁₄S₂⁺ (M)⁺: 1412.4719, found 1412.4719 (Δ3.5 ppm).

Synthesis of Dioxaborolane Bearing Pentamethine Cyanine Modified NHS Ester (2)

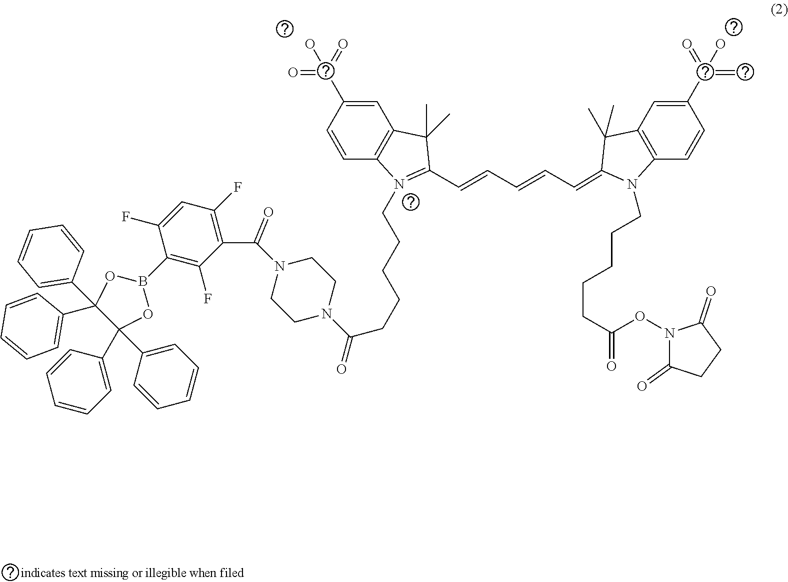

The following reagents were added to a 1.3 mL glass v-vial: CY5.18.OH (12 mg, 16 μmols), Piperazin-1-yl(2,4,6-trifluoro-3-(4,4,5,5-tetraphenyl-1,3,2-dioxaborolan-2-yl)phenyl)methanone (12 mg, 19 μmols), HOBt (14 mg, 91 μmols), 800 μL DMF, 80 μL pyridine, and EDC (150 mg, 785 μmols). The reaction proceeded for 2.5 hours at room temperature before an excess of N-hydroxy succinimide (15 mg, 130 μmols) was added. The reaction was left overnight at room temperature before it was chromatographed by preparative, reverse phase HPLC. Fractions containing 2 were lyophilized to give 2 (3.45 mg, 2.4 μmols, blue solid). The resulting solid was dissolved in DMSO and aliquot into 1 mM 20 μL aliquots which are stored at −78° C. UPLC-MS+:a10-90% H2O: ACN (0.05% TFA), 1.5 min gradient, 0.6 mL/min flow, det. 1441 M/Z, 215, 650, nm Abs spectra. Elution time: 1.79 min

Fluoridation of NHS Ester 1 and 2

Compounds 1 or 2 were reacted with 0.8 μL of 100 mM HF (80 nmols) fluoride pH 3.0, which was heated to dryness at 80° C. Dry contents were re-suspended with a 20 μL volume of water and analyzed by reverse phase HPLC-MS.

Fluoridation of Dioxaborolane Bearing Trimethine Cyanine Modified NHS Ester (1)

The fluoridation of 1 proceeds cleanly yielding only an NHS bearing fluorescent compound with absorption in the Cy3 bandwidth (550 nm).

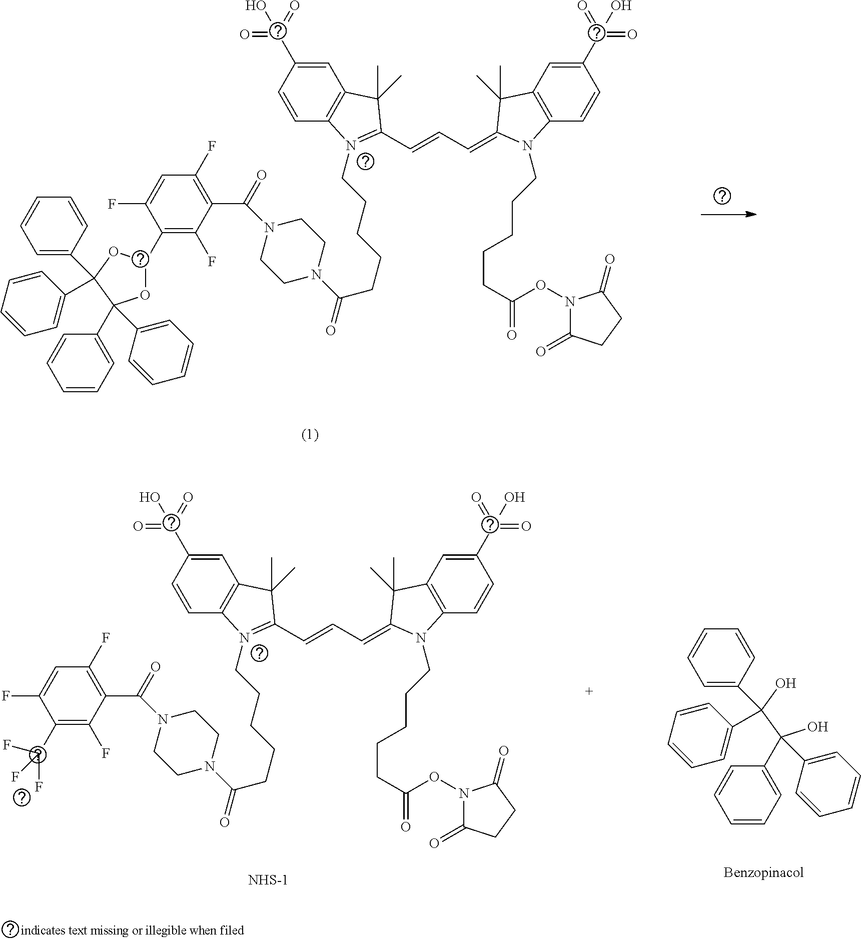

UPLC-MS⁺ a10-90% H₂O:ACN (0.05% TFA), 1.5 min gradient, 0.6 mL/min flow, det. 1088 M/Z, 550, 215 nm Abs spectra. Elution time: 1.13 min NHS-1, 1.89 min pinacol. HRMS (ESI) calculated for C₅₀H₅₄BF₅N₅O₁₂S₂⁻ (M-F)⁻: 1086.3229, found 1086.3278 (Δ4.5 ppm).

Fluoridation of Dioxaborolane Bearing Pentamethine Cyanine modified NHS Ester (2)

The fluoridation of 2 proceeds cleanly, yielding an NHS bearing fluorescent compound with absorption in the Cy5 bandwidth (650 nm).

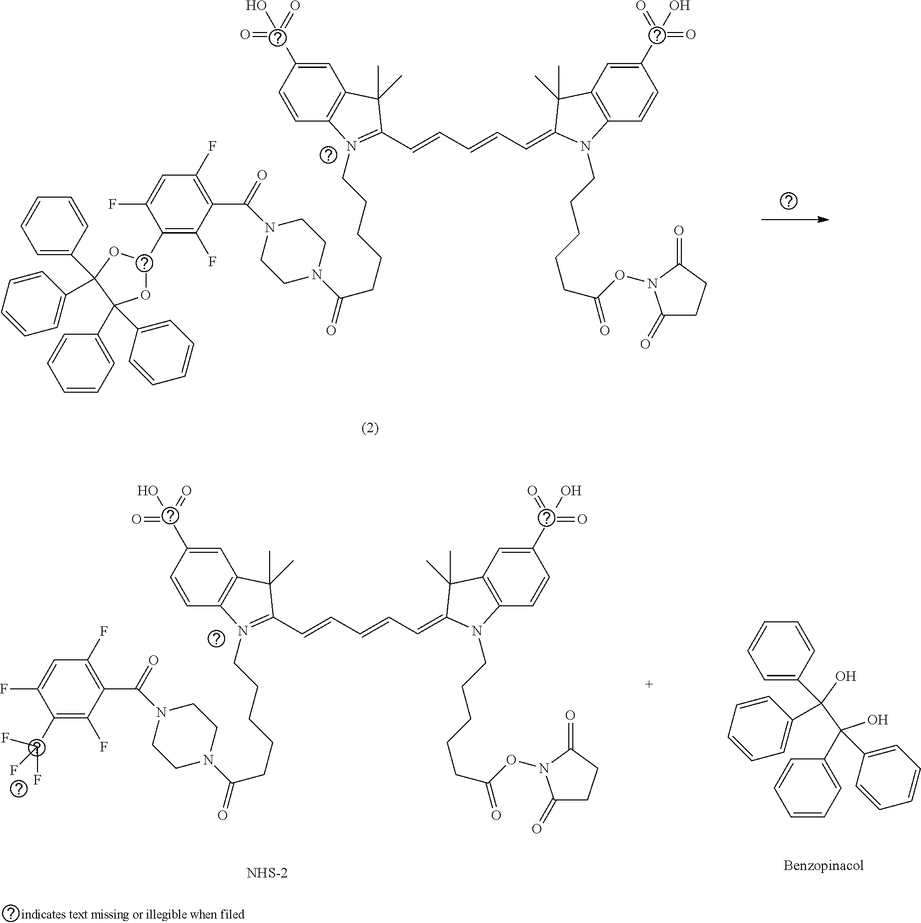

UPLC-MS⁺:a10-90% H₂O:ACN (0.05% TFA), 1.5 min gradient, 0.6 mL/min flow, det. 1114 M/Z, 550, 215 nm Abs spectra. Elution time: 1.22 min NHS-2, 1.89 min pinacol.

Radiolabeling of RBCs with 18F-PET Containing Cyanine Fluorophores 1 and 2

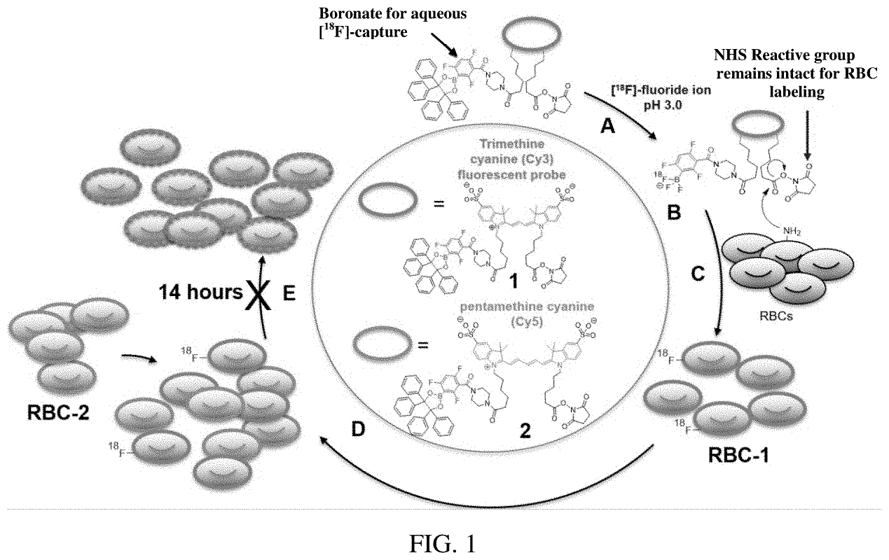

The process of labeling RBCs with the above-described fluorophore compositions 1 and 2 is schematically depicted in FIG. 1. Referring to letters A-E in FIG. 1: in step (A), a small molecule precursor is reacted with radioactive [¹⁸F] or non-radioactive [¹⁹F] fluoride, to give (B) cell-reactive material that (C) labels amines on the surface of RBCs. In step (D), cells labeled with different fluorophores can be mixed. In step (E), RBC populations do not exchange dyes (14 hours).

Notably, in a mixture of [¹⁸F]-PET/NIRF RBCs cells labeled with 1 (Cy3) and 2 (Cy5) the fluorescent label remains bound to the original RBC to which it was introduced and does not transfer to other cells even after 14 hours, as shown by the absence of observable cells in two fluorescence channels. RBCs labeled with compound 1 were mixed with RBCs that were labeled with compound 2 and left at 25° C. for 14 hours. Unfixed bright field imaging was performed on mixed RBC-1 and RBC-2 cells. RBC-1 was imaged under fluorescent conditions using 531(40) nm excitation and 593(40) nm emission filters. An overlay of fluorescent RBC-1 and RBC-2 show that there was no mixing of 1 or 2 on RBCs after 14 hours. RBC-2 was imaged using 628(40) nm excitation and 692(40) nm emission filters. An overlay of bright field and fluorescent images show that RBCs contained the original dye that they were labeled with, and that 1 or 2 were evenly distributed to RBCs.

Confirmation of RBC viability following the labeling of RBCs with [¹⁸F]-PET/NIRF probes 1 (Cy3) and 2 (Cy5).

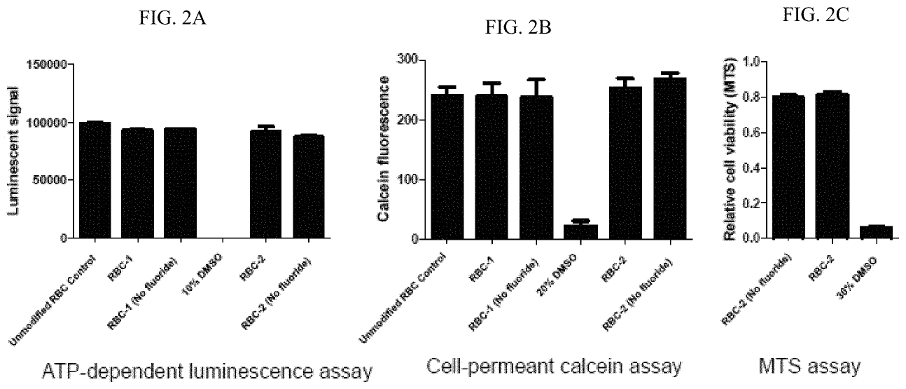

RBCs labeled with compound 1 and 2 were assayed for cell viability using commercial kits. FIGS. 2A-2C show the results for a luminescent ATP detection assay (FIG. 2A), a cell-permeant calcein assay (FIG. 2B), and a cell proliferation assay (FIG. 2C). Controls included unmodified RBCs (positive control) and non-viable RBCs that had been inactivated with DMSO (negative control). Blood (500 μL) was collected from an anesthetized BALB/C mouse by cardiac puncture in the presence of heparin as an anticoagulant. Isolated RBCs were re-suspended with 10 ml PBS, and washed 3 times with 10 ml PBS in a centrifuge at 200 rcf for 20 minutes. 1 mL aliquots of blood were distributed to six vials: Vial 1 contained 1 mL of unmodified RBCs (1.5×108 cells, positive control); Vials 2 and 4 contain 1 mL of blood and 2.5 μL of 1 or 2 which were pre-reacted with 2.5 μL of 200 mM HF for 1 hour, and neutralized with 5 μL of 10×PBS, to give RBC-1 and RBC-2; Vials 2 and 4 contain 1 mL of blood added to 2.5 μL of 1 and 2 that were not treated with fluoride. Vial 6 contained 1 mL of blood and 111 μL of DMSO (negative control). The mixtures were incubated for 30 minutes. Labeled cells were then washed with RPMI-1640, 10% FBS media, 3 times in a centrifuge set at 200 rcf for 20 minutes. All vials were re-suspended in 10 mL of medium and seeded into each well of a 96-well plate with black walls and clear bottoms (100 μL/well). An ATP-dependent luminescent cell viability assay, cell-permeant calcein AM assay, and an MTS cell proliferation assay were used to verify RBC viability on a Tecan Infinite® M1000. The ATP-dependent luminescence assay was performed with the addition of 100 μL CellTiter Glo® reagent into each well followed by incubation at room temperature for 15 minutes. Luminescence settings were used to collect data. For the Calcein AM assay, 100 μL of 4 μM Calcein AM in PBS was added into each well. The mixture was incubated at 37° C. for 30 minutes. Fluorescence was measured with an excitation wavelength of 485 nm and emission filter at 530 nm. For the MTS cell proliferation assay, 20 μL of CellTiter 96® Aqueous One reagent was added into each well. The mixture was incubated at 37° C. for 4 hours. Absorbance was measured at 490 nm.

Radiochemical Analysis of [¹⁸F]-RBC Purification by Centrifugation

Six washes of RBC (with 15 mL of PBS) were performed to remove unreacted, NHS-ester 1, [¹⁸F]-fluoride ion, and hydrolyzed 1 or 2, away from [¹⁸F]-RBC. Three different labeling conditions were performed/attempted on RBCs, as follows:

(A) NHS containing 1 was labeled with 60 mCi of [¹⁸F]-fluoride ion in a 1 to 4 ratio of 1 to fluoride. Concentration of the mixture was performed before neutralization, RBC addition, and PBS washes. The resulting [¹⁸F]-RBC activity obtained was 1.6 mCi after 6 PBS washes;

(B) To confirm that RBCs do not take up fluoride nonspecifically, NHS-1 was labeled with 71 mCi of [¹⁸F]-fluoride ion in a 1 to 3 ratio of 1 to fluoride. Concentration of this mixture to a fluoride concentration that is greater than 20 mM was not performed before neutralization and RBC addition. The resulting [¹⁸F]-RBC activity obtained was 4 μCi, 400 fold less (3.5 hour synthesis, decay uncorrected), and could not be imaged in brain in or ex vivo;

(C) The labeling of 1 was performed a third time, with 46 mCi of [¹⁸F]-fluoride ion, in the presence of a large excess of ¹⁹F carrier fluoride in a 1 to 125 ratio of 1 to fluoride. Concentration of the mixture was performed before neutralization, RBC addition, and PBS wash. The resulting [¹⁸F]-RBC activity obtained was <1 μCi.

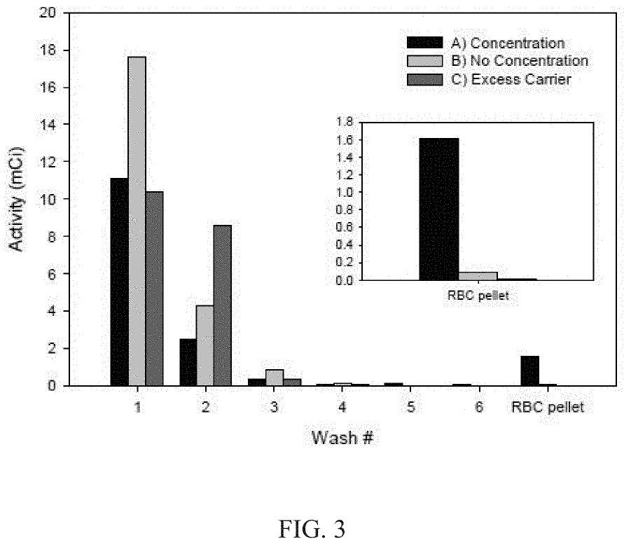

The results of conditions A-C are shown in FIG. 3. As shown in FIG. 3, only condition (A) resulted in the desired, labeled [¹⁸F]-RBC.

Fluorescence Imaging of Hemorrhage In Vivo

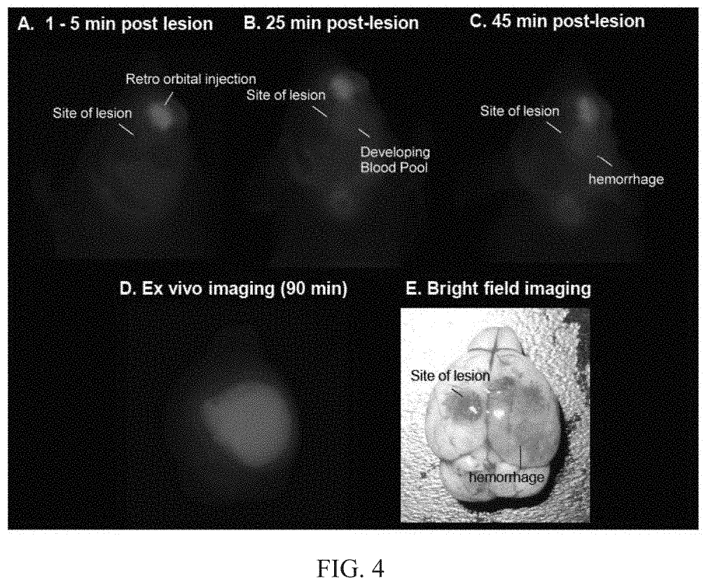

Fluorescence from [¹⁸F]-PET/NIRF 2 was used to image hemorrhage in vivo, by use of near infrared imaging of agent labeled with Cy5 dye 2. FIG. 4 shows the real time observation of traumatic progression. The following images are shown in FIGS. 4: (A) 1 to 5 minute post-lesion, (B) 25 minute post-lesion, and (C) 45 minute post-lesion in a skull-exposed cryolesion bearing mouse (note the growing blood pool), (D) ex vivo fluorescence imaging showing blood pool in the brain, and (E) bright-field imaging confirming site of lesion and hemorrhage. Notably, imaging in (E) is clearer than (A-C) due to removal of skull in (E).

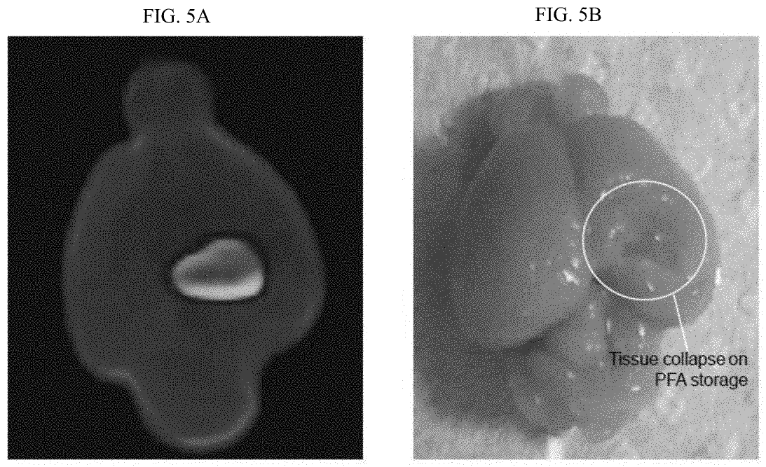

FIGS. 5A and 5B show the results for ex vivo RBC-1 [¹⁸F]-PET imaging of intracranial hemorrhage. FIG. 5A shows an ex vivo PET/CT brain image of a tail vein injection of RBC-1 of a mouse 40 minutes after cryolesion. In an attempt to preserve brain tissue for PET/MR imaging, brain tissue was preserved in 4° C., refrigerated in 4% paraformaldehyde PBS solution for a week following PET acquisition. FIG. 5B shows ex vivo bright field imaging after week-long PFA storage. FIG. 5B clearly shows a lesion corroborating the [¹⁸F]-RBC-1 signal in the PET/CT; however, macroscopic coloration corresponding to the presence of viable RBCs at the site of lesion was not present. Additionally, the region of brain tissue containing the hemorrhage had clearly disintegrated. An MR of this tissue did not provide any more meaningful data than the bright field image. For three-dimensional co-registration of PET and MR hemorrhage data, imaging must be performed on fresh tissue.

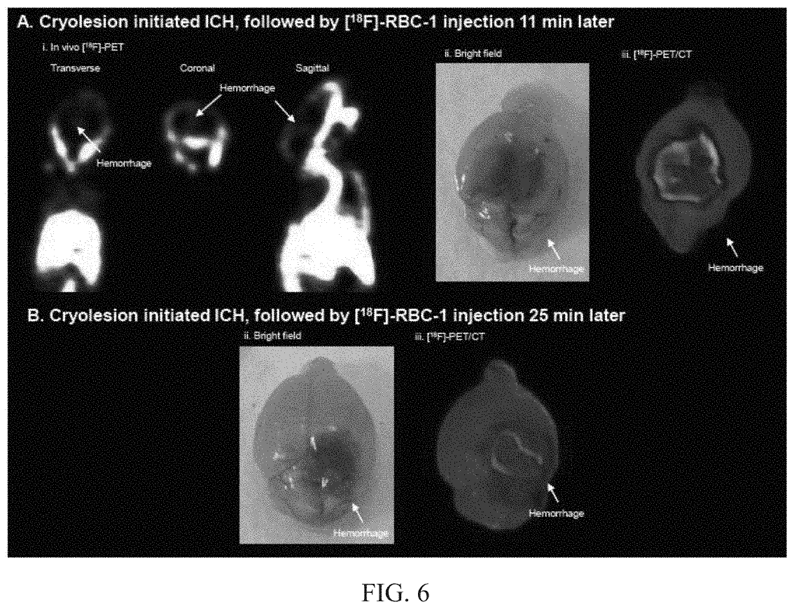

Intracranial hemorrhage was observed in vivo and ex vivo, after temporal delays were inserted between cryolesion and [¹⁸F]-RBC-1 injection. [¹⁸F]-RBC-1 was used to image intracranial hemorrhage following a delay between cryolesion and [¹⁸F]-RBC-1 injection. Cryolesions were initiated in mice under isoflurane anesthesia. Time was allowed to pass before [¹⁸F]-RBC-1 was introduced through the tail vein. The images are shown in FIG. 6 as follows: Image (Ai) shows transverse, coronal, and sagittal PET slices of a mouse that had received a cryolesion 11 min before [¹⁸F]-RBC-1 was introduced through the tail vein. Intracranial hemorrhage is indicated in each slice with white arrows. Intracranial hemorrhage was confirmed by bright field imaging (image Aii) and PET/CT imaging (image Aiii) of this brain following immediate excision after whole body image acquisition. Bright field (image Bii) and PET/CT (image Biii) imaging was used to confirm intracranial hemorrhage after a 25 minute delay was inserted between cryolesion and [¹⁸F]-RBC-1 tail vein injection.

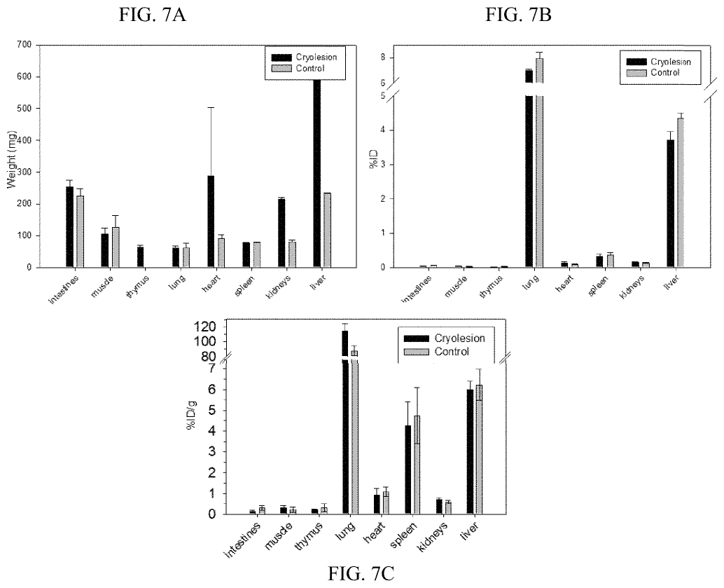

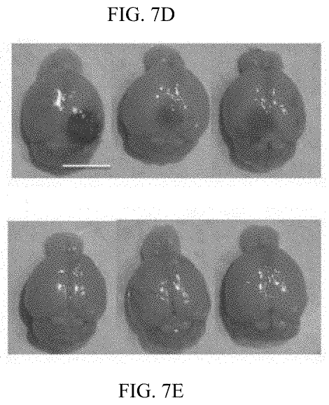

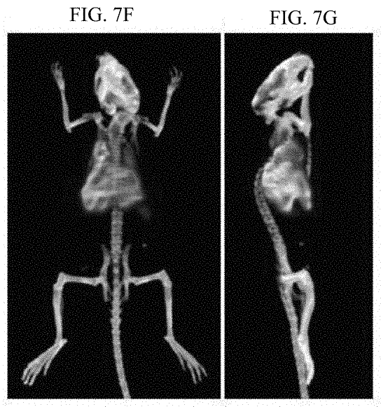

FIGS. 7A-7G show the scintillated biodistribution of [¹⁸F]-RBC-1 60 minutes after cryolesion and tail vein injection. FIG. 7A shows the general biodistribution of RBC-1 after 50 minutes following tail vein injection to lung, spleen, and liver. There was minimal localization of RBC-1 to intestines, kidneys, and muscle, which indicates that RBC-1 can be used to visualize other bleeding disorders such as intestinal bleeding, renal bleeding, and more general internal bleeding in emergency situations. Tissue weight obtained 1 hour after RBC-1 injection and cryolesion (n=3 mice with cryolesions and n=3 control mice with no cryolesion), error bars=SEM. FIGS. 7B and 7C show [¹⁸F]-scintillated biodistribution reported in percent injected dose (% ID) (FIG. 7B) and percent injected dose per gram (% ID/g) (FIG. 7C). Notably, brain hemorrhage did not significantly affect distribution of RBC-1 in other tissues (Error bars=SEM). Fresh blood for biodistribution was collected from the left ventricle of a functional heart immediately following cervical dislocation. FIG. 7D shows ex vivo bright-field imaging of brains bearing intracranial hemorrhage in cryolesion cohort. FIG. 7E shows images of brains of mice in control cohort. Notably, the brain hemorrhage did not significantly affect distribution of RBC-1 in other tissues (scale bar: 0.5 cm). FIGS. 7F and 7G show ventral and side PET/CT projections, respectively, confirming distribution data in FIGS. 7B and 7C. Images were contrasted to focus on the major organs of RBC-1 distribution in mice. Notably, the general lack of RBC-1 in the abdominal regions of mice indicates that [¹⁸F]-RBCs should be additionally useful in the imaging of intestinal bleeding, renal bleeding, and internal bleeding in emergency situations.

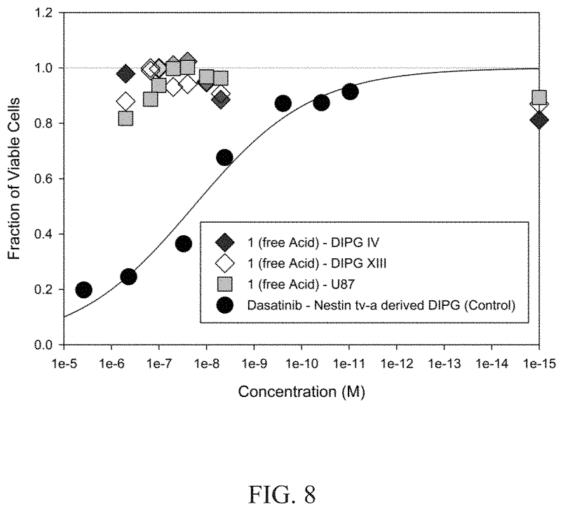

Cell viability studies were carried out as described for FIGS. 2A-2C. High-concentration solutions of 1 were incubated with different immortal glial cell lines. The fraction of viable cells are provided in the graph of FIG. 8. As shown by the results in FIG. 8, toxicity was not observed when high concentrations of 1 were incubated with for 48 hours or more with DIPG IV, DIPG XIII, or U87 cell lines. A reference consisting of dasatinib in Nestin tv-a derived DIPG is shown as a control IC₅₀ 19±20 nM, R²=0.972 four parameter logistic standard curve regression analysis. Toxicity due to 1 was not observed. Thus, the results indicate that [¹⁸F]-RBC-1 does not trigger glial toxicity.

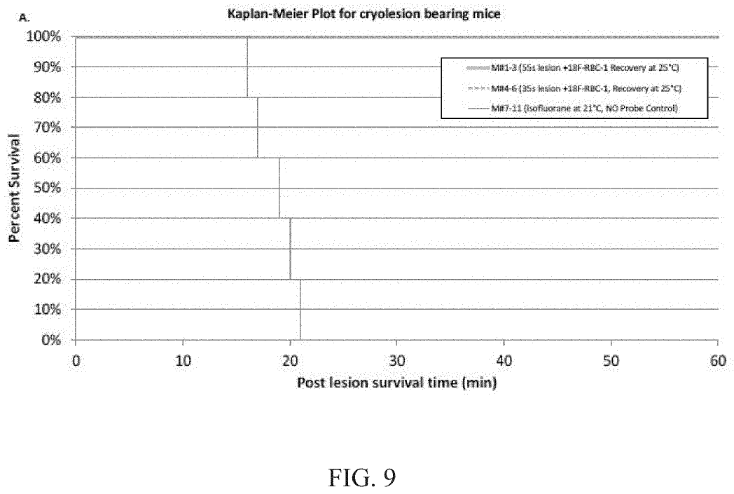

FIG. 9 is a Kaplan-Meier Plot showing that cryolesion/PET associated hypothermia can be avoided with recovery between cryolesion and scanning Mortality due to cryolesion-related hypothermia can occur in the bore of an Inveon™ PET/CT calibrated at 21° C. A warmed recovery step must be implemented between cryolesion and imaging to avoid mortality (M #1-3, M #4-6). If mice are immediately transferred from the operating table to the bore of an Inveon PET/CT (calibrated at 21° C.) following cryolesion, death can occur (M #7-11). The following experiment identifies hypothermic death due to cryolesion (and not toxicity of 1) if cryolesion is directly proceeded by PET/CT imaging, without a recovery step. Note that morbidity can also be reduced by reducing cryoprobe contact time.

Three cohorts of mice were prepared. Cohort A, consisting of 3 mice (M #1, 2, 3) were anesthetized with isoflurane and exposed to a cryoprobe for 55 s (100 g of pressure, 7.9 g/mm², over a 12.6 mm² contact area). Mice in Cohort B (3 mice, M #4, 5, 6) were anesthetized and exposed to shorter, 35 s, cryolesion contact times focused on the right posterior cerebral cortex Immediately following cryolesion, these 6 mice (Cohort A and B) were injected with [¹⁸F]-RBC-1 (tail vein), disconnected from isoflurane anesthesia, and were immediately transferred to a cage heated to 25° C. using a temperature-controlled space heater, where all 6 mice were allowed to recover. All 6 mice survived to the point of deliberate sacrifice, for longer than 3 hours at 25° C. Photography taken between 1-3 hours show viable, cryolesion bearing mice. A control cohort, Cohort C, consisting of 5 mice, were anesthetized and were treated with 55 s cryolesions focused on the right posterior cerebral cortex. This cohort, Cohort C, was not injected with any agent, i.e. these mice received cryolesion but no PET injectate (no-probe control). Following cryolesion, Cohort C mice were maintained under isoflurane and immediately transferred into the bore of an Inveon PET/CT that was calibrated at 21° C. The Kaplan-Meier plot (shown in FIG. 9) demonstrates that all post-cryolesion mice in cohort C died between 15 and 25 minutes in the bore of the PET scanner. Mortality from cryolesion-PET/CT imaging is due to cryolesion-related hypothermia. Mortality is not related to the injected [¹⁸F]-RBC-1 imaging agent. Mortality can be eliminated by allowing mice to recover in a warmed cage immediately following cryolesion. Mice receiving cryolesions with a reduced contact time (Cohort B, 35 s) showed smaller volume lesions in PET/CT analyses vs. Cohort C, where cryoprobe contact time is longer (55 s).

In other experiments, intracranial hemorrhage was observed with [¹⁸F]-RBC-1-PET 50 to 157 minutes after cryolesion, after longer temporal delays (12 to 107 minutes) were inserted between cryolesion. Specifically, longer temporal delays were inserted between cryolesion and contrast agent injection (longer delays are used vs. FIG. 6, where 11 and 25 minute delays were implemented). Following cryolesion (55 seconds), mice were immediately transferred to a heated cage at 25° C. without anesthesia to prevent mortality from hypothermia. The following times were allowed to pass before [¹⁸F]-RBC-1 was introduced through the tail vein: (A) 107 min, (B) 95 min, (C) 30 min, (D) 14 min, and (E) 12 min. Mice were sacrificed 40 minutes following injection. Brains were resected and imaged by (i) brightfield and (ii) PET (orange)/CT imaging. Artifacts associated with tissue excision are sometimes observed in sectional analyses. e.g., bleeding in olfactory bulb that may or may not be related to cryolesion, and bone fragment and blood accumulation at the base of the brain possibly from ex vivo drainage following organ excision.

Synthesis of Compound B

First, Cur-BF₂ was prepared according to the following scheme and synthesis:

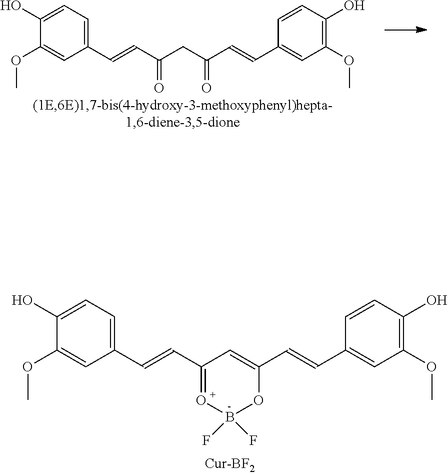

Curcumin (1106 mg, 3 mmol) was dissolved in 10 mL of dry CH₂Cl₂.BF₃.(OEt)₂ (46.5%, 916 mg, 6 mmol) was added to the solution dropwise, under magnetic stirring at room temperature. The reaction was monitored with UPLC. After two hours of reaction, 5 mL of deionized water was added to quench the reaction. The mixture was dried by rotary evaporation. Dried material was re-dissolved in DMF (HPLC) and purified by preparative chromatography (Agilent, 1260 Infinity) equipped with a C18 Reversed Phase LC Column (Luna® 10 μm C18, 100 Å, LC Column 250×21.2 mm). The gradient was water/acetonitrile at a flow rate of 12 mL/min (90/10 to 10/90 linear gradient, 20 min, and then isocratic water/acetonitrile 10/90 for another 25 min). Ultrapure water and HPLC acetonitrile (Sigma, St. Louis, Mo., USA) were used. The fractions of 19 min to 20 min were collected, kept at −80° C. overnight and then lyophilized into red color powder. Yields: 717 mg (57.4%). ¹H NMR (500 MHz, d6-DMSO, 25° C., TMS): δ 10.10 (s, 2H), 7.92 (d, J=15.6 Hz, 2H, isomer, trans), 7.73 (d, J=8.6 Hz, 2H, isomer, cis), 7.47 (d, J=1.8 Hz, 2H), 7.34 (dd, J=8.3 Hz, 2H), 7.02 (d, J=15.6 Hz, 2H), 6.88 (d, J=8.2 Hz, 2H), 6.45 (s, 1H), 3.85 (s, 6H); ¹³C NMR (125 MHz, d6-DMSO, 25° C., TMS): δ 179.19, 151.81, 148.64, 147.42, 126.45, 125.70, 118.33, 116.42, 112.89, 101.55, 56.23; ¹⁹F NMR (470 MHz, d6-DMSO, 25° C., CFCl₃): δ A major product (A) and a minor isomer (B) are observed. (A) [¹⁰B]-BF₂: δ -140.361 (34.7%), [¹¹B]-BF₂: δ -140.423 (65.3%) (Δ0.062 ₚₚₘ), (B) [¹⁰B]-BF₂: δ -140.230 (16.7%), [¹¹B]-BF₂: δ -140.295 (83.3%) (Δ0.065 ppm); HRMS (ESI) calc'd for [M]=[C₂₁H₁₉BF₂O₆]: 416.1243, calc'd for [M−H]⁻=[C₂₁H₁₈BF₂O₆]⁻: 415.1165, found [M−H]⁻: 415.1164.

Next, 6F-Cur-BF2 was prepared according to the following scheme and synthesis:

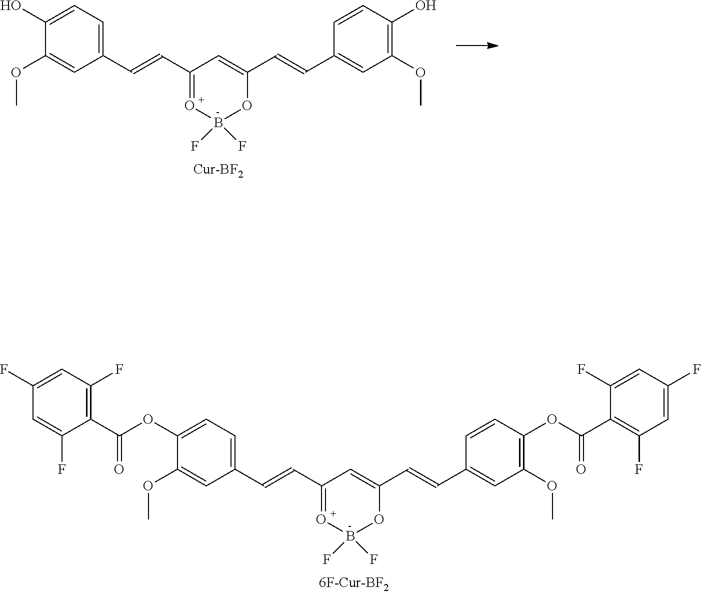

Cur-BF₂ (12.5 mg, 0.03 mmol), 2,4,6-Trifluorobenzoic acid (15.8 mg, 0.09 mmol), HOBt hydrate (12.2 mg, 0.09 mmol), EDC hydrochloride (57.6 mg, 0.3 mmol) and 20 μL pyridine were dissolved in 1 mL DMF (HPLC, Sigma) in a 1.5 mL eppendorf tube. The reaction, shielded from light, was left at room temperature overnight. A color change from weak orange-red to bright green is visible under 365 nm UV lamp illumination indicating reaction. The product was isolated by preparative chromatography (Agilent, 1260 Infinity) equipped with a C18 Reversed Phase LC Column (Luna® 10 pm C18, 100 Å, LC Column 250×21.2 mm). The gradient was water/acetonitrile at a flow rate of 12 mL/min (50/50 to 0/100 linear gradient, 20 min, and then isocratic water/acetonitrile 0/100 for another 25 min). Ultrapure water and HPLC acetonitrile (Sigma, St. Louis, Mo., USA) were used. The fractions of 21 min to 22 min were collected, kept at −80° C. overnight and then lyophilized into yellow color powder. Yields: 17.2 mg (78.3%). ¹H NMR (500 MHz, d6-DMSO, 25° C., TMS): δ 8.10 (d, J=15.8 Hz, 2H, isomer, trans), 8.02 (d, J=8.4 Hz, 2H, isomer, cis), 7.76 (s, 2H), 7.57 (d, J=8.15 Hz, 2H), 7.48 (t, J=9.25 Hz, 4H), 7.39 (d, J=8.15 Hz, 2H), 7.34 (d, J=15.75 Hz, 2H), 6.68 (s, 1H), 3.90 (s, 6H); ¹³C NMR (125 MHz, d6-DMSO, 25° C., TMS): δ 180.62, 164.15, 162.98, 162.91, 162.85, 162.78, 160.93, 160.86, 160.80, 160.73, 158.25, 151.52, 146.67, 141.83, 134.24, 131.66, 123.85, 123.38, 122.58, 114.13, 106.25, 106.22, 102.98, 102.95, 102.77, 102.74, 102.56, 102.53, 56.80; ¹⁹F NMR (470 MHz, d6-DMSO, 25° C., CFCl₃): δ -102.030 (t, J=10.25 Hz, 2F), −107.581 (t, J=10 Hz, 4F), −139.407 (2F, A major product (A) and a minor isomer (B) are observed. (A) [¹⁰B]-BF₂: δ -139.445 (30.6%), [¹¹B]-BF₂: δ -139.508 (69.4%) (Δ0.063 ppm), (B) [¹⁰B]-BF₂: δ -139.296 (11.1%), [¹¹B]-BF₂: δ -139.369 (88.9%) (Δ0.073 ppm)); HRMS (ESI) calc'd for [M]=[C₃₅H₂₁BF₈O₈]: 732.1202, found [M]⁻: 732.1185.

Cur-BF2-Mal was also prepared according to the following scheme and synthesis:

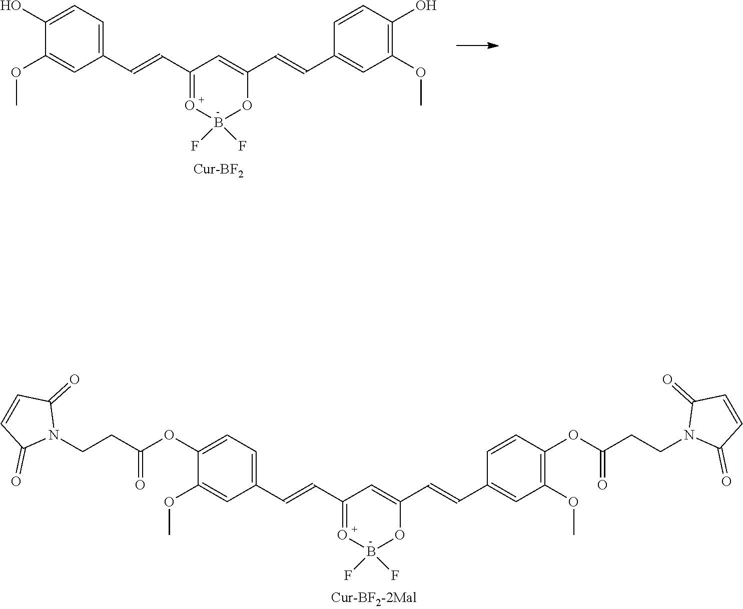

Cur-BF₂ (12.5 mg, 0.03 mmol), 3-Maleimidopropionic acid (15.2 mg, 0.09 mmol), HOBt hydrate (12.2 mg, 0.09 mmol), EDC hydrochloride (57.6 mg, 0.3 mmol) and 20 μL pyridine were dissolved in 1 mL DMF (HPLC, Sigma) in a 1.5 mL eppendorf tube. The reaction, shielded from light, was left at room temperature overnight. A color change from weak orange-red to bright green that is visible under 365 nm UV lamp illumination indicates reaction. The product was isolated by preparative chromatography (Agilent, 1260 Infinity) equipped with a C18 Reversed Phase LC Column (Luna® 10 μm C18, 100 Å, LC Column 250×21.2 mm). The gradient was water/acetonitrile at a flow rate of 12 mL/min (90/10 to 0/100 linear gradient, 20 min, and then isocratic water/acetonitrile 0/100 for another 25 min). Ultrapure water and HPLC acetonitrile (Sigma, St. Louis, Mo., USA) were used. The fractions of 20 min to 21 min were collected, kept at −80° C. overnight and then lyophilized into yellow color powder. Yields: 7.2 mg (33.4%). ¹H NMR (500 MHz, d6-DMSO, 25° C., TMS): δ 8.06 (d, J=15.7 Hz, 2H, isomer, trans), 7.95 (d, J=8.5 Hz, 2H, isomer, cis), 7.66 (s, 2H), 7.51 (d, J=7.55 Hz, 2H), 7.29 (d, J=15.8 Hz, 2H), 7.22 (d, J=8.2 Hz, 2H), 7.07 (d, J=3.0 Hz, 4H), 6.65 (s, 1H), 3.83 (s, 6H), 3.78 (t, J=7.0 Hz, 4H), 2.91 (t, J=7.0 Hz, 4H); ¹³C NMR (125 MHz, d6-DMSO, 25° C., TMS): δ 180.53, 171.27, 171.21, 168.99, 151.65, 146.79, 142.40, 135.17, 133.67, 123.94, 123.29, 123.13, 122.26, 113.78, 102.67, 56.63, 56.60, 33.66, 32.57; ¹⁹F NMR (470 MHz, d6-DMSO, 25° C., CFCl₃): A major product (A) and a minor isomer (B) are observed. (A) [¹⁰B]-BF2: δ -139.550 (30.1%), [¹¹B]-BF₂: δ -139.614 (69.9%) (Δ0.063 ppm), (B) [¹⁰B]-BF₂: δ -139.418 (15.4%), [¹¹B]-BF₂: δ -139.483 (84.6%) (Δ0.065 ppm). HRMS (ESI) calc'd for [M]=[C₃₅H₂₉BF₂N₂O₁₂]: 718.1782, found [M]−: 718.1759.

Proof of Radiolabeling Compound B

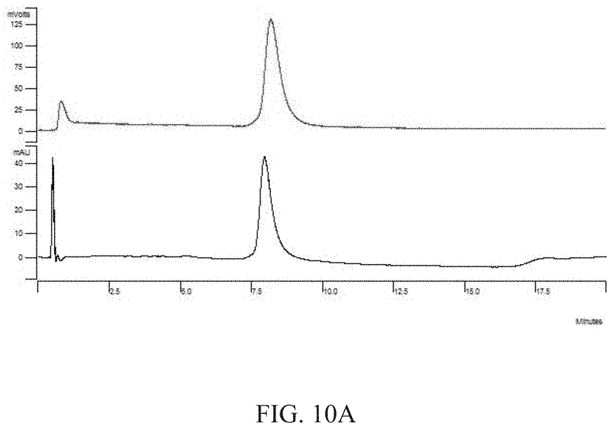

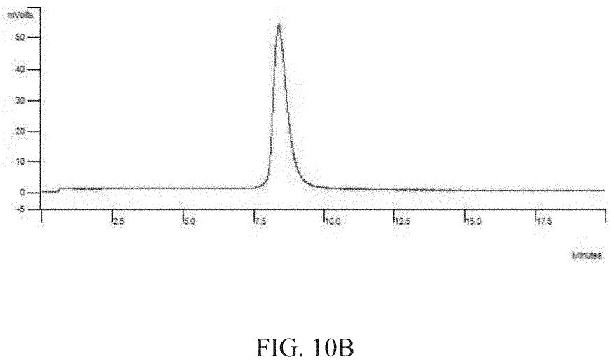

6F-Cur-BF₂: A 10 minute, a60-100 linear gradient was used to characterize 6F-Cur-BF₂ radiolabeling. UV-Vis absorbance at 450 nm was used to monitor 6F-Cur-BF₂ elution. FIG. 10A shows the UV-Vis trace (bottom) and radioactivity trace (top). As shown in FIG. 10A, 6F-Cur-BF₂ elutes at 8.1982 min and correlates with the radioactivity trace. Unreacted, contaminating fluoride-18 ion is visible at 1 min in the radiotrace.

To isolate [¹⁸F]-6F-Cur-BF₂, water was added to give a precipitate containing pure [¹⁸F]-6F-Cur-BF₂. Supernatant containing fluoride-18 ion was decanted. The radioactivity trace of purified [¹⁸F]-6F-Cur-BF₂ in DMSO is shown in FIG. 10B.

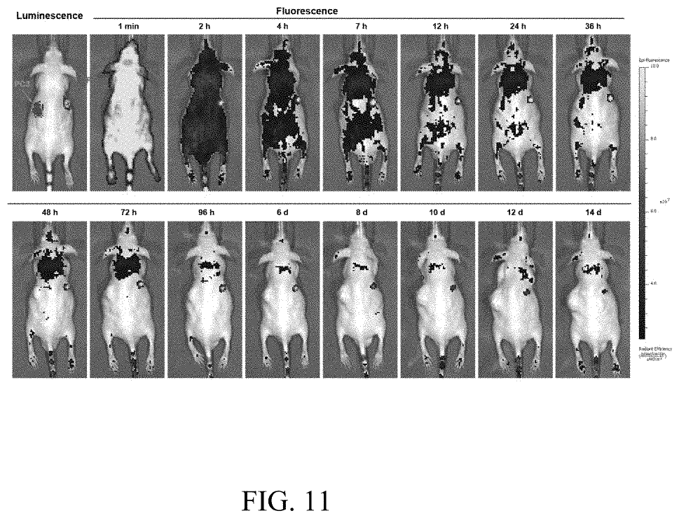

FIG. 11. presents murine data showing that a 18F-labeled-PSMA-targeted-[¹⁸F]-PET/NIRF (Cy3) contrast agent remains fluorescent long after 18F has decayed. A mouse is injected with a positron-emitting and fluorescent PSMA-targeted agent. PSMA tissue retains >50% of its maximum fluorescent intensity for at least 14 days post-injection. The mouse had contralateral PSMA- (left flank) and PSMA⁺ (right flank) xenografts. These data are evidence that a patient can receive a single injection of the present PET/Fluorescent agent, have a ¹⁸F PET/CT or PET/MR scan, and then receive a delayed fluorescence-guided radical prostatectomy.

FIGS. 12A and 12B present human Cy3-fluorescent prostate histopathology showing that the present [¹⁸F]-PET/FL agent would be useful regarding human PSMA⁺ biopsies. A patient received IV 0.15 mg of PSMA⁺-targeted-[¹⁸F]-PET/NIRF (Cy3) (120 nmol) ˜1h prior to receiving a standard-of-care prostate cancer biopsy. Tissue was collected, sectioned (frozen), and analyzed by confocal microscopy. FIG. 12A shows Cy3 fluorescence (pink)/DAPI (nuclear stain-blue) histology with pink PSMA⁺ glands. FIG. 12B shows confirmative PSMA⁺ immunohistochemical staining in a neighboring section. Note that PET/FL-Cy3 fluorescence (pink in A) corroborate glandular PSMA⁺ IHC (brown in FIG. 12B). PET/FL-Cy3 fluorescence is only present in abnormal gland bearing PSMA⁺ tissue.

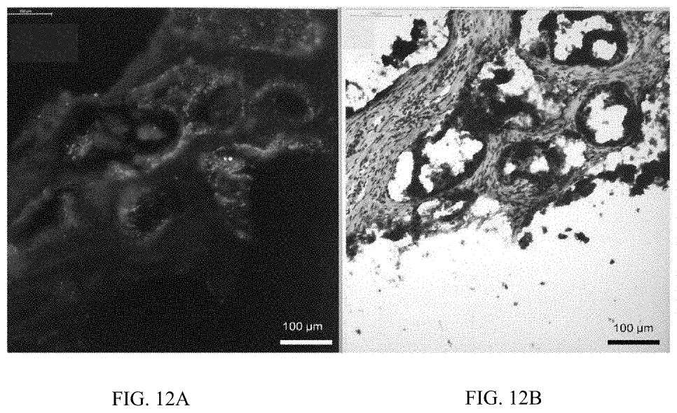

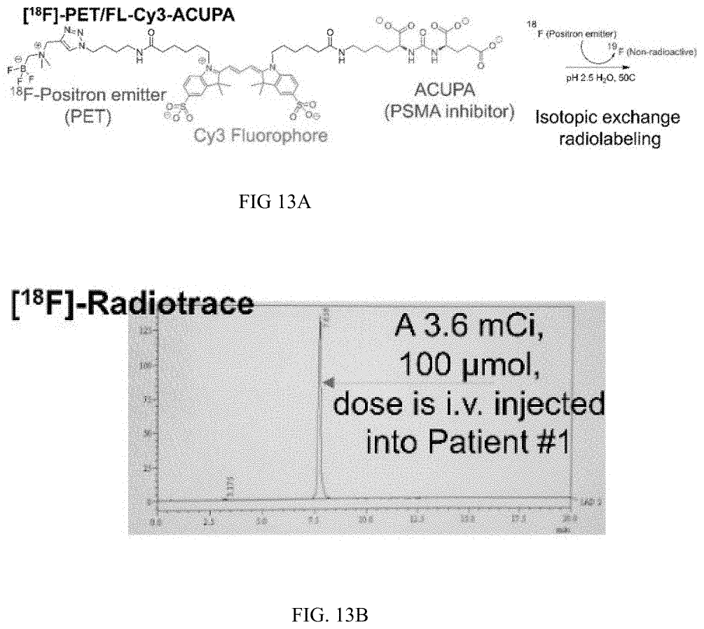

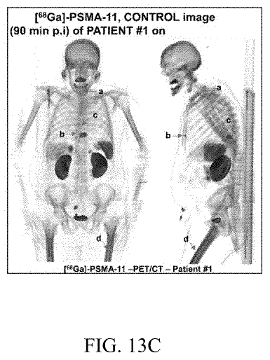

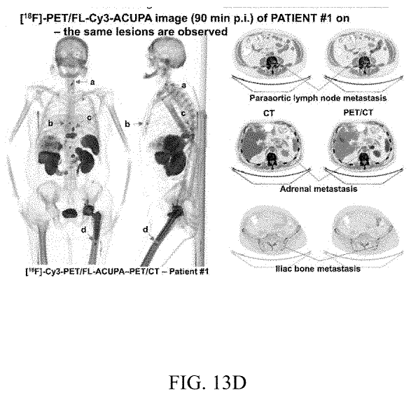

FIGS. 13A-13D provide preliminary data showing that the present [¹⁸F]-PET/FL agent has use in humans. Patient #1 has a case of disseminated PSMA⁺ cancer. FIG. 13A shows that PSMA⁺-targeted-[¹⁸F]-PET/NIRF (Cy3) was ¹⁸F-radiolabeled (FIG. 13B), and injected into the right cephalic vein of Patient #1. FIG. 13C shows that an existing [⁶⁸Ga]-PSMA-11 PET (MIP) scan served as a control for Patient #1 (90 min p.i., Gleeson >8, biopsy-confirmed-PSMA⁺, PET (red) CT (grey)). FIG. 13D shows a PET scan with [¹⁸F]-PET/NIRF (Cy3) (8 months later, 90 min p.i., 3.6 mCi, <100 μmol) reveals the same major lesions (a-d). Patient #1 received [¹⁷⁷Lu]-PSMA-617 therapy and hip replacement surgery in between the scans shown in FIG. 13C and FIG. 13D. [¹⁸F]-PET/FL-Cy3 PET shows advanced, disseminated disease, and suggests that Patient #1 is not a candidate for radical prostatectomy.

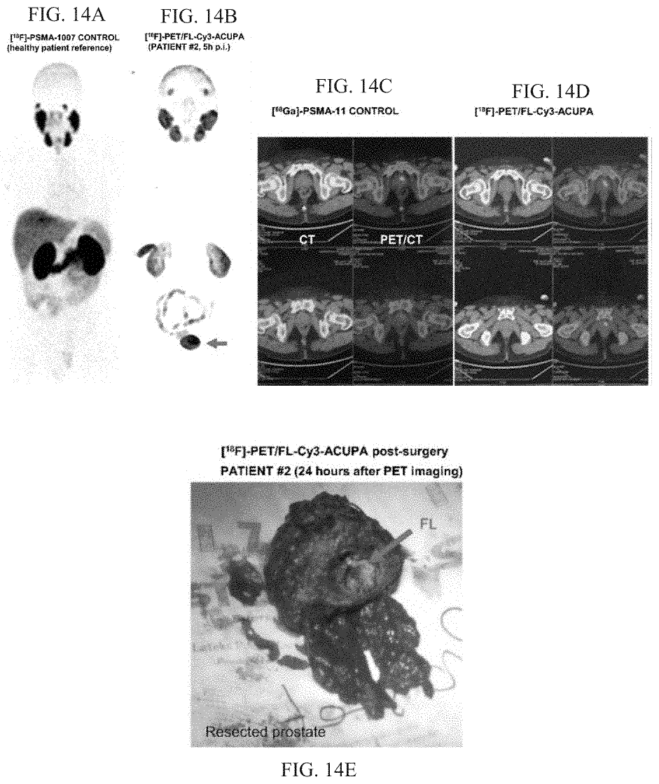

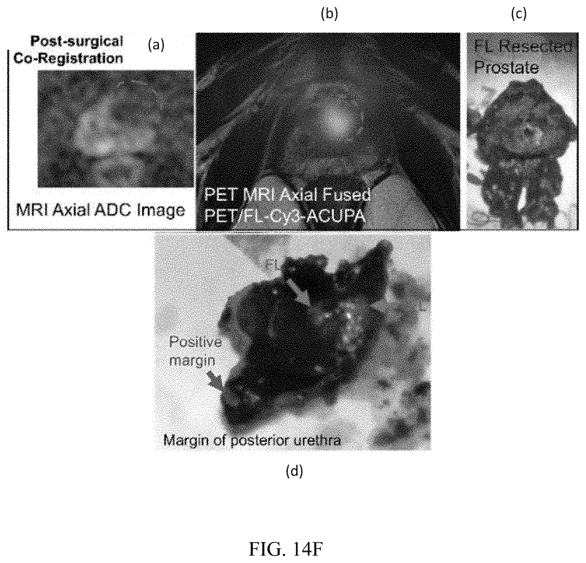

FIGS. 14A-14F present preliminary data in a second patient showing that the present [¹⁸F]-PET/FL agent has PET use regarding human biopsies and intraoperative radical prostatectomy (back table intraoperative consult). Patient #2 has localized PSMA⁺ cancer and is a candidate for PSMA-targeted-[¹⁸F]-PET/NIRF (Cy3)-guided radical prostatectomy. FIG. 14A Control, A [¹⁸F]-PSMA-1007 PET (MIP) image in a healthy, non-cancerous patient is like the one in FIG. 14B, showing a [¹⁸F]-PET/FL (Cy3) PET (MIP) scan of Patient #2 (5 mCi, MIP, 5h p.i.). Patient #2 has localized PSMA⁺ prostate cancer that had not disseminated from the prostate (arrow). FIG. 14C is a [⁶⁸Ga]-PSMA-11 scan of Patient #2 which confirmed the PET scan in FIG. 14B revealing localized PSMA⁺ disease and no lymph node involvement. Localized prostate cancer is indicated with an arrow. FIG. 14D shows that the same PET delineation is observed in FIG. 14B (a [⁶⁸Ga]-PSMA-11 scan) and in FIG. 14D (a [¹⁸F]-PET/FL (Cy3) scan performed one month later (D=90 min p.i.)). Patient #2 is an ideal candidate for radical prostatectomy. Without undergoing re-injection, Patient #2 underwent radical prostatectomy 24 hours later (after ¹⁸F decay). FIG. 14E shows that resected tissues were fluorescent and PSMA⁺. FIG. 14F shows that post-surgical prostate image co-registration shows prostate cancer in MRI-ADC, PET, and FL. Unfortunately, post-surgical fluorescence imaging reveals that the urologist left a positive (un-resected) margin in the posterior urethra (red arrow).

While there have been shown and described what are at present considered the preferred embodiments of the invention, those skilled in the art may make various changes and modifications which remain within the scope of the invention defined by the appended claims.

What is claimed is:

1. A method for internal imaging of biological tissue in a subject by positron emission tomography (PET) or single photon emission computer tomography (SPECT), the method comprising:
(i) administering to said subject an imaging agent comprising the following structure: 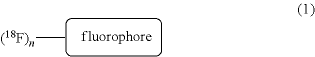
wherein n is an integer of at least 1, and the one or more fluorine-I 8 (¹⁸E) radionuclides in Formula (1) are bound directly or indirectly to the fluorophore; and
(ii) imaging internal biological tissue of said subject by PET or SPECT.

2. The method of claim 1, wherein said imaging agent further comprises a biological agent bound to the fluorophore, wherein the imaging agent has the following structure: 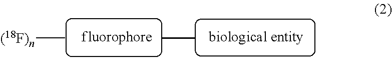
wherein said biological entity is selected from blood cell, peptide, nucleotide, aptamer, targeting agent, antibody, and antibody fragment.

3. The method of claim 1, wherein said imaging agent is administered intravenously.

4. The method of claim 1, further comprising simultaneously imaging said internal biological tissue by fluorescence imaging,

5. The method of claim 1, wherein said internal imaging is used to assess or monitor transplant rejection or acceptance.

6. The method of claim 1, wherein said internal imaging is used to assess or monitor the extent or progression of a cancer.

7. The method of claim 6, wherein said cancer is prostate cancer.

8. The method of claim 6, wherein said cancer is breast cancer.

9. The method of claim 1, wherein said internal imaging is used to assess or monitor a hemorrhage.

10. The method of claim 9, wherein said hemorrhage is in the brain.

11. The method of claim 2, wherein said biological entity is a blood cell, and the imaging agent has the following structure:

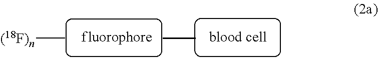

12. The method of claim 11, wherein said fluorophore is directly or indirectly covalently bound to said blood cells.

13. The method of claim 11, wherein said blood cells are red blood cells.

4. The method of claim 11, wherein said blood cells are selected from white blood cells and platelets.

15. The method of claim 1, wherein said fluorophore is an organofluorophore containing at least one carbon-carbon bond and at least one carbon-hydrogen bond.

16. The method of claim 15, wherein said fluorophore is a cyanine-based fluorophore.

17. The method of claim 16, wherein said cyanine-based fluorophore contains at least three conjugated carbon-carbon double bonds, at least one of which is not in a ring.

18. The method of claim 15, wherein said fluorophore is a xanthene-based fluorophore.

19. The method of claim 18, wherein said xanthene-based fluorophore contains a fluorescein structure.

20. The method of claim 15, wherein said fluorophore contains at least two pyrrolyl rings.

21. The method of claim 1, wherein the fluorine-18 radionuclide is bound to a boron atom that is bound directly or indirectly to said fluorophore.

22. The method of claim 21, wherein said fluorine-18 radionuclide is part of a —BF₂— linking group or a —BF₃ terminal group.

23. The method of claim 15, wherein said fluorophore contains a polyalkyleneoxide group that contains at least two alkyleneoxide units.

24. The method of claim 15, wherein said fluorophore contains at least one sulfonic acid or sulfonate salt group.

25. The method of claim 1, wherein the method is directed to imaging of prostate cancer tissue.

26. The method of claim 2, wherein the method is directed to imaging of prostate cancer tissue.

27. The method of claim 26, wherein the imaging agent has the following structure: 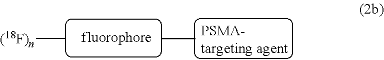
wherein PSMA is prostate-specific membrane antigen and said PSMA-targeting agent is a molecule that selectively targets PSMA.

28. The method of claim 26, wherein said fluorophore is a Cy3 cyanine dye.

29. The method of claim 27, wherein said fluorophore is a Cy3 cyanine dye.

30. The method of claim 28, wherein the method provides an extended imaging window.

31. The method of claim 27, wherein said imaging agent has the following structure: 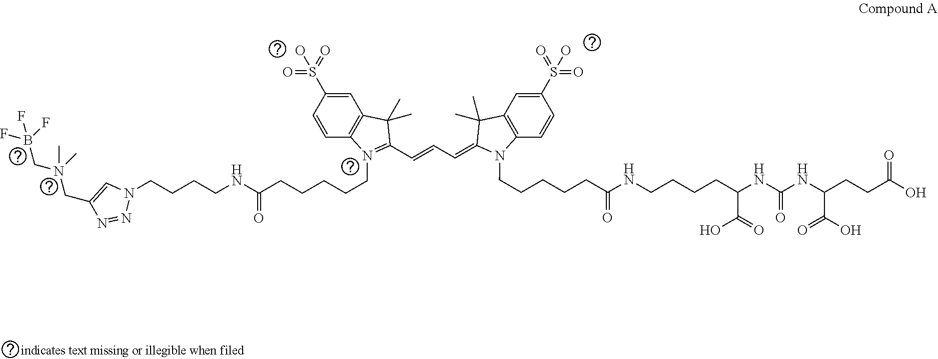
wherein the 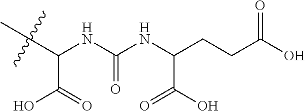 [SMILES: *C(NC(=O)NC(CCC(=O)O)C(=O)O)C(=O)O]
moiety is a PSMA-targeting agent.

32. The method of claim 1, wherein the method is directed to imaging of a lymph node.

33. The method of claim 32, wherein the imaging agent according to Formula (1) has the following structure:

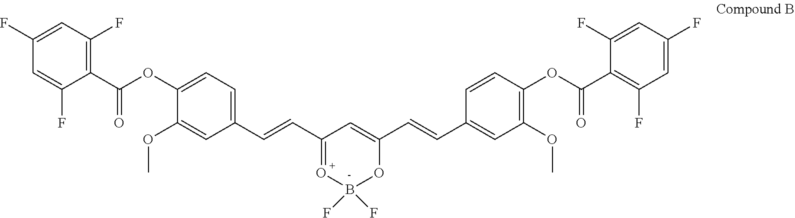

34. The method of claim 1, wherein the method is directed to imaging of cerebral spinal

35. The method of claim 34, wherein the imaging agent according to Formula (1) has the following structure: 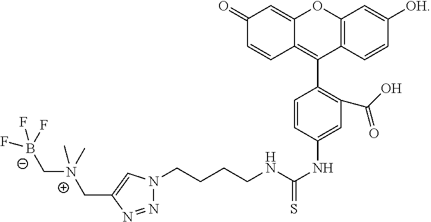 [SMILES: C[N+](C)(Cc1cn(CCCCNC(=S)Nc2ccc(-c3c4ccc(=O)cc-4oc4cc(O)ccc34)c(C(=O)O)c2)nn1)C[B-](F)(F)F]
